(19) United States
(12) Patent Application Publication
(10) Pub. No.: US 2005/0266099 A1
(43) Pub. Date: Dec. 1, 2005

(54) Methods and apparatus for modifying properties of the bbb and cerebral circulation by using the neuroexcitatory and/or neuroinhibitory effects of odorants on nerves in the head

(76) Inventor: Alon Shalev, Ra' anana (IL)

Correspondence Address: DARBY & DARBY P.C., P. O. BOX 5257, NEW YORK, NY, 10150-5257, US

(21) Appl. No.: 10/512,780

(22) PCT Filed: Apr. 25, 2003
(86) PCT No.: PCT/IL03/00338

§ 371 Date: Jun. 1, 2005

Related U.S. Application Data
(60) Provisional application No. 60/376,048, filed on Apr. 25, 2002.
(60) Provisional application No. 60/461,232, filed on Apr. 8, 2003.

(51) Int. Cl. A61K 35/78

(52) U.S. Cl. 424/725

(57) ABSTRACT

A method for modifying a property of a brain of a patient includes presenting an odorant to an air passage of the patient, the odorant having been selected for presentation to the air passage because it is such as to increase conductance of molecules from a systemic blood circulation of the patient through a blood brain barrier (BBB) of the brain into brain tissue of the patient. The molecules are selected from the group consisting of: a pharmacological agent, a therapeutic agent, an endogenous agent, and an agent for facilitating a diagnostic procedure.

### CROSS-REFERENCES TO RELATED APPLICATIONS

This application claims priority from U.S. Provisional Patent Application 60/376,048 to Shalev, entitled, “Methods and apparatus for modifying properties of the BBB and cerebral circulation by using the neuroexcitatory and/or neuroinhibitory effects of odorants on nerves in the head,” filed Apr. 25, 2002, which is assigned to the assignee of the present patent application and is incorporated herein by reference.

This application also claims priority from a US provisional patent application to Gross et al., filed Apr. 8, 2003, entitled, “Treating abnormal conditions of the mind and body by modifying properties of the blood-brain barrier and cephalic blood flow,” which is assigned to the assignee of the present patent application and is incorporated herein by reference.

### FIELD OF THE INVENTION

The present invention relates generally to medical procedures and electronic devices. More specifically, the invention relates to the use of electrical devices for implantation in the head, for example, in the nasal cavity. The invention also relates to methods for using odorants to induce or to inhibit neural activity for the treatment of a clinical condition. The invention also relates to apparatus and methods for administering drugs, for treating stroke and headaches such as migraine and cluster headaches, and for improving cerebral blood flow.

### BACKGROUND OF THE INVENTION

The blood-brain barrier (BBB) is a unique feature of the central nervous system (CNS) which isolates the brain from the systemic blood circulation. To maintain the homeostasis of the CNS, the BBB prevents access to the brain of many substances circulating in the blood.

The BBB is formed by a complex cellular system of endothelial cells, astroglia, pericytes, perivascular macrophages, and a basal lamina. Compared to other tissues, brain endothelia have the most intimate cell-to-cell connections: endothelial cells adhere strongly to each other, forming structures specific to the CNS called “tight junctions” or zonula occludens. They involve two opposing plasma membranes which form a membrane fusion with cytoplasmic densities on either side. These tight junctions prevent cell migration or cell movement between endothelial cells. A continuous uniform basement membrane surrounds the brain capillaries. This basal lamina encloses contractile cells called pericytes, which form an intermittent layer and probably play some role in phagocytosis activity and defense if the BBB is breached. Astrocytic end feet, which cover the brain capillaries, build a continuous sleeve and maintain the integrity of the BBB by the synthesis and secretion of soluble growth factors (e.g., gamma-glutamyl transpeptidase) essential for the endothelial cells to develop their BBB characteristics.

Because of the BBB, certain non-surgical treatments of the brain based upon systemic introduction of compounds through the bloodstream have been ineffective or less effective. For example, chemotherapy has been relatively ineffective in the treatment of CNS metastases of systemic cancers (e.g., breast cancer, small cell lung cancer, lymphoma, and germ cell tumors), despite clinical regression and even complete remission of these tumors in non-CNS systemic locations. The most important factors determining drug delivery from blood into the CNS are lipid solubility, molecular mass, and electrical charge. A good correlation exists between the lipid solubility of a drug, expressed as the octanol/water partition coefficient, and the drug's ability to penetrate or diffuse across the BBB. This is particularly relevant for drugs with molecular weights smaller than 600 dalton (Da). The normal BBB prevents the passage of ionized water soluble drugs with a molecular weight greater than 180 Da. Most currently-available effective chemotherapeutic agents, however, have a molecular weight between 200 and 1200 Da. Therefore, based both on their lipid solubilities and molecular masses, the passage of many agents is impeded by the BBB.

In addition to transcellular diffusion of lipophilic agents, there are several specific transport mechanisms to carry certain molecules across the brain's endothelial cells. Specific transport proteins exist for required molecules, such as glucose and amino acids. Additionally, absorptive endocytosis and transcytosis occur for cationized plasma proteins. Specific receptors for certain proteins, such as transferrin and insulin, mediate endocytosis and transport across the cell.

Non-surgical treatment of neurological disorders is generally limited to systemic introduction of compounds such as neuropharmaceuticals and other neurologically-active agents that might remedy or modify neurologically-related activities and disorders. Such treatment is limited, however, by the relatively small number of known compounds that pass through the BBB. Even those that do cross the BBB often produce adverse reactions in other parts of the body or in non-targeted regions of the brain.

There have been a number of different studies regarding efforts to cross the BBB—specifically, with regard to overcoming the limited access of drugs to the brain. Such efforts have included, for example, chemical modification, development of more hydrophobic analogs, or linking an active compound to a specific carrier. Transient opening of the BBB in humans has been achieved by intracarotid infusion of hypertonic mannitol solutions or bradykinin analogs. Also, modulation of the P-glycoprotein, whose substrates are actively pumped out of brain cells into capillary lumens, has been found to facilitate the delivery of drugs to the brain. However, due to the inherent limitations of each of the aforementioned procedures, there is still a heed for more generic, effective, and predictable ways to cross the BBB.

It would also be desirable to develop controllable means for modulating cerebral blood flow. Many pathological conditions, such as stroke, migraine, and Alzheimer's disease, are significantly affected or exacerbated by abnormal cerebral blood flow.

U.S. Pat. No. 5,756,071 to Mattem et al., which is incorporated herein by reference, describes a method for nasally administering aerosols of therapeutic agents to enhance penetration of the blood brain barrier. The patent describes a metering spray designed for pernasal application, the spray containing at least one sex hormone or at least one metabolic precursor of a sex hormone or at least one derivative of a sex hormone or combinations of these, excepting the precursors of testosterone, or at least one biogenic amine, with the exception of catecholamines.

U.S. Pat. No. 5,752,515 to Jolesz et al., which is incorporated herein by reference, describes apparatus for image-guided ultrasound delivery of compounds through the blood-brain barrier. Ultrasound is applied to a site in the brain to effect in the tissues and/or fluids at that location a change detectable by imaging. At least a portion of the brain in the vicinity of the selected location is imaged, e.g., via magnetic resonance imaging, to confirm the location of that change. A compound, e.g., a neuropharmaceutical, in the patient's bloodstream is delivered to the confirmed location by applying ultrasound to effect opening of the blood-brain barrier at that location and, thereby, to induce uptake of the compound there.

The following references, which are incorporated herein by reference, may be useful:
Delepine L, Aubineau P, “Plasma protein extravasation induced in the rat dura mater by stimulation of the parasympathetic sphenopalatine ganglion,” Experimental Neurology, 147, 389-400 (1997)
Hara H, Zhang Q J, Kuroyanagi T, Kobayashi S, “Parasympathetic cerebrovascular innervation: An anterograde tracing from the sphenopalatine ganglion in the rat,” Neurosurgery, 32, 822-827 (1993)
Jolliet-Riant P, Tillement J P, “Drug transfer across the blood-brain barrier and improvement of brain delivery,” Fundam. Clin. Pharmacol., 13, 16-25 (1999)
Kroll R A, Neuwelt E A, “Outwitting the blood brain barrier for therapeutic purposes: Osmotic opening and other means,” Neurosurgery, 42, 1083-1100 (1998)
Sanders M, Zuurmond W W, “Efficacy of sphenopalatine ganglion blockade in 66 patients suffering from cluster headache: A 12-70 month follow-up evaluation,” Journal of Neurosurgery, 87, 876-880 (1997)
Syelaz J, Hara H, Pinard E, Mraovitch S, MacKenzie E T, Edvinsson L, “Effects of stimulation of the sphenopalatine ganglion on cortical blood flow in the rat,” Journal of Cerebral Blood Flow and Metabolism,” 8, 875-878 (1988)
Van de Waterbeemd H, Camenisch G, Folkers G, Chretien J R, Raevsky O A, “Estimation of blood brain barrier crossing of drugs using molecular size and shape and h bonding descriptors,” Journal of Drug Targeting,” 6, 151-165, (1998)
Suzuki N, Hardebo J E, Kahrstrom J, Owman C, “Selective electrical stimulation of postganglionic cerebrovascular parasympathetic nerve fibers originating from the sphenopalatine ganglion enhances cortical blood flow in the rat,” Journal of Cerebral Blood Flow and Metabolism, 10, 383-391 (1990)
Suzuki N, Hardebo J E, Kahrstrom J, Owman C H, “Effect on cortical blood flow of electrical stimulation of trigeminal cerebrovascular nerve fibres in the rat,” Acta Physiol. Scand., 138, 307-315 (1990)
Major A, Silver W, “Odorants presented to the rat nasal cavity increase cortical blood flow,” Chem. Senses, 24, 665-669 (1999)
Fusco B M, Fiore G, Gallo F, Martelletti P, Giacovazzo M, “‘Capsaicin-sensitive’ sensory neurons in cluster headache: pathophysiological aspects and therapeutic indications,” Headache, 34, 132-137 (1994)
Lambert G A, Bogduk N, Goadsby P J, Duckworth J W, Lance J W, “Decreased carotid arterial resistance in cats in response to trigeminal stimulation,” Journal of Neurosurgery, 61, 307-315 (1984)
Silver W L, “Neural and pharmacological basis for nasal irritation,” in Tucker W G, Leaderer B P, Molhave L, Cain WS (eds), Sources of Indoor Air Contaminants, Ann. NY Acad. Sci., 641, 152-163 (1992)
Silver W, “Chemesthesis: the burning questions,” ChemoSense, Vol. 2 No. 1, 1-2 (1999)

### OBJECTS OF THE INVENTION

It is an object of some aspects of the present invention to provide improved methods and apparatus for delivery of compounds to the brain, particularly through the BBB.

It is also an object of some aspects of the present invention to provide such methods and apparatus as can be employed to deliver such compounds through the BBB with a minimally invasive approach.

It is a further object of some aspects of the present invention to provide such methods and apparatus as can facilitate delivery of large molecular weight compounds through the BBB.

It is yet a further object of some aspects of the present invention to provide cost-effective methods and apparatus for delivery of compounds through the blood-brain-barrier.

It is still a further object of some aspects of the present invention to provide improved methods and apparatus for remedying or modifying neurological activities and disorders via delivery of compounds through the blood-brain-barrier.

It is also a further object of some aspects of the present invention to modulate cerebral blood flow.

It is an additional object of some aspects of the present invention to provide improved methods and apparatus for treating stroke.

It is yet an additional object of some aspects of the present invention to provide improved methods and apparatus for treating migraine, cluster and other types of headaches.

It is still an additional object of some aspects of the present invention to provide improved methods and apparatus for treating neurological diseases (for example, Alzheimer's disease), whose prognosis and evolution of pathological symptoms are influenced by cerebral blood flow.

It is also an object of some aspects of the present invention to provide implantable apparatus which affects a property of the brain, without actually being implanted in the brain. In particular, the apparatus may be implanted in the nasal cavity.

It is a further object of some aspects of the present invention to provide methods which affect a property of the brain without the use of implantable apparatus. In particular, the methods may comprise presenting odorants to an air passage of a patient, such as a nasal cavity or the throat.

It is yet a further object of some aspects of the present invention to affect a property of the brain by using the neuroexcitatory and/or neuroinhibitory effects of odorants on nerves in the head.

These and other objects of the invention will become more apparent from the description of preferred embodiments thereof provided hereinbelow.

### SUMMARY OF THE INVENTION

In preferred embodiments of the present invention, an electrical stimulator drives current into the sphenopalatine ganglion (SPG) or into neural tracts originating or reaching the SPG. Typically, the stimulator drives the current in order to control and/or modify SPG-related behavior, e.g., in order to induce changes in cerebral blood flow and/or to modulate permeability of the blood-brain barrier (BBB). These embodiments may be used in many medical applications, such as, by way of illustration and not limitation, (a) the treatment of cerebrovascular disorders such as stroke, (b) the treatment of migraine, cluster and other types of headaches, or (c) the facilitation of drug transport across the BBB.

It is to be appreciated that, whereas preferred embodiments of the present invention are described with respect to driving current into the SPG or into neural structures directly related thereto, the scope of the present invention includes driving current into other sites in the brain which upon stimulation modulate cerebral blood flow or modulate permeability properties of the BBB, as appropriate for a given application.

It is also to be appreciated that electrical “stimulation,” as provided by preferred embodiments of the present invention, is meant to include substantially any form of current application to designated tissue, even when the current is configured to block or inhibit the activity of nerves.

It is further to be appreciated that implantation and stimulation sites, methods of implantation, and parameters of stimulation are described herein by way of illustration and not limitation, and that the scope of the present invention includes other possibilities which would be obvious to someone of ordinary skill in the art who has read the present patent application.

It is yet further to be appreciated that while preferred embodiments of the invention are generally described herein with respect to electrical transmission of power and electrical stimulation of tissue, other modes of energy transport may be used as well. Such energy includes, but is not limited to, direct or induced electromagnetic energy, RF transmission, ultrasonic transmission, optical power, and low power laser energy (via, for example, a fiber optic cable).

It is additionally to be appreciated that whereas preferred embodiments of the present invention are described with respect to application of electrical currents to tissue, this is to be understood in the context of the present patent application and in the claims as being substantially equivalent to applying an electrical field, e.g., by creating a voltage drop between two electrodes.

The SPG is a neuronal center located in the brain behind the nose. It consists of parasympathetic neurons innervating the middle cerebral and anterior cerebral lumens, the facial skin blood vessels, and the lacrimal glands. Activation of this ganglion is believed to cause vasodilation of these vessels. A second effect of such stimulation is the opening of pores in the vessel walls, causing plasma protein extravasation (PPE). This effect allows better transport of molecules from within these blood vessels to surrounding tissue.

The middle and anterior cerebral arteries provide the majority of the blood supply to the cerebral hemispheres, including the frontal and parietal lobes in their entirety, the insula and the limbic system, and significant portions of the following structures: the temporal lobes, internal capsule, basal ganglia and thalamus. These structures are involved in many of the neurological and psychiatric diseases of the brain, and preferred embodiments of the present invention are directed towards providing improved blood supply and drug delivery to these structures.

There is also some animal evidence for the presence of SPG-originated parasympathetic innervation in the posterior cerebral and basilar arteries. Consistent with the assumption that this is also the case in humans, many regions of the human brain are within the reach of treatments provided by preferred embodiments of the present invention, as described hereinbelow.

Currently the SPG is a target of manipulation in clinical medicine, mostly in attempted treatments of severe headaches such as cluster headaches. The ganglion is blocked either on a short-term basis, by applying lidocaine, or permanently, by ablation with a radio frequency probe. In both cases the approach is through the nostrils. In some preferred embodiments of the present invention, similar methods for approaching the SPG are utilized, to enable the electrical stimulation or electrical blocking thereof.

According to a preferred embodiment of the instant invention, a method and apparatus are provided to enhance delivery of therapeutic molecules across the BBB by stimulation of the SPG and/or its outgoing parasympathetic tracts and/or another parasympathetic center. The apparatus typically stimulates the parasympathetic nerve fibers of the SPG, thereby inducing the middle and anterior cerebral arteries to dilate, and also causing the walls of these cerebral arteries to become more permeable to large molecules. In this manner, the movement of large pharmaceutical molecules from within blood vessels to the cerebral tissue is substantially increased. Preferably, therefore, this method can serve as a neurological drug delivery facilitator, without the sacrifices in molecular weight required by techniques of the prior art. In general, it is believed that substantially all pharmacological treatments aimed at cerebral cells for neurological and psychiatric disorders are amenable for use with these embodiments of the present invention. In particular, these embodiments may be adapted for use in the treatment of disorders such as brain tumors, epilepsy, Parkinson's disease, Alzheimer's disease, multiple sclerosis, schizophrenia, depression, stress, obesity, pain, anxiety, and any other CNS disorders that are directly or indirectly affected by changes in cerebral blood flow or by BBB permeability changes.

Advantageously (and even in the absence of BBB permeability changes), patients with these and other disorders are generally helped by the vasodilation secondary to stimulation of the SPG, and the resultant improvement in oxygen supply to neurons and other tissue. For some applications, this treatment is given on a long-term basis, e.g., in the chronic treatment of Alzheimer's patients. For other applications, the treatment is performed on a short-term basis, e.g., to minimize the damage following an acute stroke event and initiate neuronal and therefore functional rehabilitation.

Blocking of nerve transmission in the SPG or in related neural tracts is used in accordance with some preferred embodiments of the present invention to treat or prevent migraine headaches.

Alternatively or additionally, the changes induced by electrical stimulation as described hereinabove are achieved by presenting odorants to an air passage of a patient, such as a nasal cavity or the throat. There is animal evidence that some odorants, such as propionic acid, cyclohexanone, and amyl acetate, significantly increase cortical blood flow when presented to the nasal cavity. This has been interpreted by some researchers as evidence that these odorants (e.g., environmental pollutants) may be involved in the formation of various headaches by increasing cerebral blood flow. The temporal profile and other quantitative characteristics of such odorant stimulation are believed by the present inventors to have a mechanism of action that has a neuroanatomical basis overlapping with that of the electrical stimulation of the SPG. Furthermore, experimental animal evidence collected by the inventors and described in U.S. Provisional Patent Application 60/368,657 to Shalev and Gross entitled, “SPG stimulation,” filed Mar. 28, 2002, which is assigned to the assignee of the present invention and is incorporated herein by reference, suggest a correlation between the mechanisms of increasing cerebral blood flow and increased cerebrovascular permeability. It is hypothesized that such increased cerebral blood flow caused by odorants is a result of stimulation of parasympathetic and/or trigeminal fibers. These fibers may mediate cerebral blood flow changes directly, by communicating with the SPG, or by some other mechanism. It is also hypothesized that these odorants stimulate via reflex arcs the SPG or other autonomic neural structures that innervate the cerebrovascular system. Therefore, the inventors hypothesize, odorant “stimulation” may increase cerebral blood flow in general, and cortical blood flow in particular, by some or all of the same mechanisms as electrical stimulation, as described hereinabove. Alternatively, odorants may cause increased cortical blood flow by other mechanisms, such as by entering the blood stream and reaching the affected blood vessels in the brain or by parasympathetic stimulation via the olfactory nerve. In addition to the effect on cerebral blood flow, the introduction of odorants into an air passage is also expected to induce an increase in the permeability of the anterior two thirds of the cerebrovascular system to circulating agents of various sizes, i.e., to increase the permeability of the BBB. Similarly, presenting certain other odorants to an air passage decreases cerebral blood flow and decreases the permeability of the BBB.

Odorants that may increase or decrease cerebral blood flow and/or the permeability of the BBB include, but are not limited to, propionic acid, cyclohexanone, amyl acetate, acetic acid, citric acid, carbon dioxide, sodium chloride, ammonia, menthol, alcohol, nicotine, piperine, gingerol, zingerone, allyl isothiocyanate, cinnamaldehyde, cuminaldehyde, 2-propenyl/2-phenylethyl isothiocyanate, thymol, and eucalyptol.

For some applications, delivery across the BBB of a pharmacological agent is enhanced by presenting an odorant to an air passage of a patient, such as a nasal cavity or the throat. In the context of the present patent application and in the claims, a pharmacological agent is an agent, for administration to a patient, that is made using pharmacological procedures. Pharmacological agents may thus include, by way of illustration and not limitation, therapeutic agents and agents for facilitating diagnostic procedures.

According to a preferred embodiment of the instant invention, a method is provided to enhance delivery of therapeutic molecules across the BBB by presenting an odorant to an air passage of a patient, such as a nasal cavity or the throat. In a preferred application, this method serves as a neurological drug delivery facilitator. The odorant is preferably presented using apparatus known in the art, such as aqueous spray nasal inhalers; metered dose nasal inhalers; or air-dilution olfactometers. Alternatively or additionally, the odorant is presented by means of an orally-dissolvable capsule that releases the active odorants upon contact with salivary liquids. The odorants reach the appropriate neural structures and induce vasodilatation, vasoconstriction and/or cerebrovascular permeability changes. Delivery of a drug can be achieved by mixing the drug with the odorant; by intravenously, intraperitoneally, or intramuscularly administering the drug, or by other delivery methods known in the art. For some applications, it is desirable to combine a local analgesic with the odorant in order to diminish any possible sensation of pain or discomfort that may directly or indirectly (e.g., via a reflex arc) accompany the odorant action upon nerves in the head. For example, preventing neural transmission in the neighboring pain fibers may be performed as a “pre-odorant” treatment, by topical administration of capsaicin together with a local analgesic for several days prior to the use of odorant stimulation. In this manner, the odorants typically induce the SPG-related response with a reduced or eliminated sensation of pain or discomfort.

In general, it is believed that substantially all pharmacological treatments aimed at cerebral cells for neurological and psychiatric disorders are amenable for use with these embodiments of the present invention. In particular, this embodiment may be adapted for use in the treatment of disorders such as brain tumors, epilepsy, Parkinson's disease, Alzheimer's disease, multiple sclerosis, schizophrenia, depression, stress, anxiety, obesity, pain, disorders requiring the administration of various growth factors, and other CNS disorders that are directly or indirectly affected by changes in cerebral blood flow or by BBB permeability changes.

Alternatively or additionally, a method is provided for increasing or reducing cortical blood flow and/or inducing or inhibiting vasodilation (even in the absence of BBB permeability changes) by presenting an odorant to an air passage of a patient, such as a nasal cavity or the throat, for treatment of a condition. Patients with the aforementioned disorders and other disorders are generally helped by vasodilation and the resultant improvement in oxygen supply to neurons and other tissue. For some applications, this treatment is given on a long-term basis, e.g., in the chronic treatment of Alzheimer's patients. For other applications, the treatment is performed on a short-term basis, e.g., to minimize the damage following an acute stroke event and initiate neuronal and therefore functional rehabilitation. Alternatively or additionally, the method provided above can be used for diagnostic purposes or in conjunction with other diagnostic methods and/or apparatus known in the art, in order to enhance diagnostic results, reduce procedure risk, reduce procedure time, or otherwise improve such diagnostic procedures and/or diagnostic results. For example, methods and apparatus described herein may be used to increase the uptake into the brain of a radio-opaque material, in order to facilitate a CT scan.

Decreasing cerebral blood flow by presenting certain odorants to an air passage is used in accordance with some preferred embodiments of the present invention to treat or prevent various types of headaches, especially with an autonomic nervous system (ANS) etiology, such as migraine and cluster headaches.

Typically, for any of the odorant presentation applications described herein, a suitable dosage of the odorant is determined for a desired application (e.g., increasing or decreasing BBB permeability, or increasing or decreasing cerebral blood flow). The procedure for determine the suitable dosage is typically performed in accordance with standard drug dosage determination procedures known in the art, e.g., testing a range of very small doses for safety and efficacy, and subsequently increasing the magnitude of the doses as safety remains acceptable and efficacy continues to increase.

There is therefore provided, in accordance with a preferred embodiment of the present invention, apparatus for modifying a property of a brain of a patient, including:
one or more electrodes, adapted to be applied to a site selected from a group of sites consisting of: a sphenopalatine ganglion (SPG) of the patient and a neural tract originating in or leading to the SPG; and
a control unit, adapted to drive the one or more electrodes to apply a current to the site capable of inducing an increase in permeability of a blood-brain barrier (BBB) of the patient.

There is also provided, in accordance with a preferred embodiment of the present invention, apparatus for modifying a property of a brain of a patient, including:
one or more electrodes, adapted to be applied to a site selected from a group of sites consisting of: a sphenopalatine ganglion (SPG) of the patient and a neural tract originating in or leading to the SPG; and
a control unit, adapted to drive the one or more electrodes to apply a current to the site capable of inducing an increase in cerebral blood flow of the patient.

There is further provided, in accordance with a preferred embodiment of the present invention, apparatus for modifying a property of a brain of a patient, including:
one or more electrodes, adapted to be applied to a site selected from a group of sites consisting of: a sphenopalatine ganglion (SPG) of the patient and a neural tract originating in or leading to the SPG; and
a control unit, adapted to drive the one or more electrodes to apply a current to the site capable of inducing a decrease in cerebral blood flow of the patient.

There is still further provided, in accordance with a preferred embodiment of the present invention, apparatus for modifying a property of a brain of a patient, including:
one or more electrodes, adapted to be applied to a site selected from a group of sites consisting of: a sphenopalatine ganglion (SPG) of the patient and a neural tract originating in or leading to the SPG; and
a control unit, adapted to drive the one or more electrodes to apply a current to the site capable of inhibiting parasympathetic activity of the SPG.

Preferably, the one or more electrodes are adapted for a period of implantation in the patient greater than about one month.

In a preferred embodiment, the apparatus includes a wire, adapted to connect the control unit to the one or more electrodes, wherein the control unit is adapted to drive the one or more electrodes from a position external to the patient.

Alternatively or additionally, the control unit is adapted to drive the one or more electrodes by wireless communication from a position external to the patient. In a preferred embodiment, the apparatus includes an electromagnetic coupling, adapted to couple the control unit and the one or more electrodes. Alternatively or additionally, the control unit is adapted to be in electro-optical communication with the one or more electrodes. Further alternatively or additionally, the control unit is adapted to be in electro-acoustic communication with the one or more electrodes. Still further alternatively or additionally, the control unit is adapted to be implanted in a nasal cavity of the patient.

Preferably, the one or more electrodes are adapted to be implanted in a nasal cavity of the patient. For some applications, at least one of the one or more electrodes includes a flexible electrode, adapted for insertion through a nostril of the patient and to extend therefrom to the site.

The apparatus preferably includes at least one biosensor, adapted to measure a physiological parameter of the patient and to generate a signal responsive thereto. The control unit, in turn, is preferably adapted to modify a parameter of the applied current responsive to the signal. As appropriate, the biosensor may include one or more of the following:
a blood flow sensor.
a temperature sensor.
a chemical sensor.
an ultrasound sensor.
transcranial Doppler (TCD) apparatus.
laser-Doppler apparatus.
a systemic blood pressure sensor.
an intracranial blood pressure sensor.
a detecting element adapted to be fixed to a cerebral blood vessel, and wherein the control unit is adapted to analyze the signal to detect an indication of a change in blood pressure indicative of a clot.
a kinetics sensor (in this case, the control unit is typically adapted to analyze the signal to detect an indication of a change in body disposition of the patient).
an electroencephalographic (EEG) sensor.
a blood vessel clot detector.

In a preferred embodiment, the control unit is adapted to configure the current so as to facilitate uptake of a drug through the BBB when the permeability of the BBB is increased.

Alternatively or additionally, the control unit is adapted to configure the current so as to increase a diameter of a blood vessel and allow an embolus that is located at a site in the blood vessel to move from the site in the blood vessel.

Further alternatively or additionally, the control unit is adapted to drive the one or more electrodes to apply the current responsive to an indication of stroke.

Still further alternatively or additionally, the control unit is adapted to drive the one or more electrodes to apply the current responsive to an indication of migraine of the patient.

There is also provided, in accordance with a preferred embodiment of the present invention, a method for modifying a property of a brain of a patient, including:
selecting a site from a group of sites consisting of: a sphenopalatine ganglion (SPG) of the patient and a neural tract originating in or leading to the SPG; and
applying a current to the site capable of inducing an increase in permeability of a blood-brain barrier (BBB) of the patient.

There is additionally provided, in accordance with a preferred embodiment of the present invention, a method for modifying a property of a brain of a patient, including:
selecting a site from a group of sites consisting of: a sphenopalatine ganglion (SPG) of the patient and a neural tract originating in or leading to the SPG; and
applying a current to the site capable of inducing an increase in cerebral blood flow of the patient.

There is yet additionally provided, in accordance with a preferred embodiment of the present invention, a method for modifying a property of a brain of a patient, including:
selecting a site from a group of sites consisting of: a sphenopalatine ganglion (SPG) of the patient and a neural tract originating in or leading to the SPG; and
applying a current to the site capable of inducing a decrease in cerebral blood flow of the patient.

There is still additionally provided, in accordance with a preferred embodiment of the present invention, a method for modifying a property of a brain of a patient, including:
selecting a site from a group of sites consisting of: a sphenopalatine ganglion (SPG) of the patient and a neural tract originating in or leading to the SPG; and
applying a current to the site capable of inhibiting parasympathetic activity of the SPG.

For some applications, the one or more electrodes are adapted for a period of implantation in the patient less than about one week.

There is further provided, in accordance with a preferred embodiment of the present invention, vascular apparatus, including:
a detecting element, adapted to be fixed to a blood vessel of a patient and to generate a signal responsive to energy coming from the blood vessel; and
a control unit, adapted to analyze the signal so as to determine an indication of an embolus in the blood vessel.

Preferably, the detecting element includes an energy transmitter and an energy receiver. For example, the energy transmitter may include an ultrasound transmitter or a transmitter of electromagnetic energy.

There is yet further provided, in accordance with a preferred embodiment of the present invention, a method for detecting, including:
fixing a detecting element to a blood vessel of a patient;
generate a signal responsive to energy coming from the blood vessel; and
analyzing the signal so as to determine an indication of an embolus in the blood vessel.

There is still further provided, in accordance with a preferred embodiment of the present invention, a method for modifying a property of a brain of a patient, including presenting an odorant to an air passage of the patient, the odorant having been selected for presentation to the air passage because it is such as to increase conductance of molecules between a systemic blood circulation of the patient and brain tissue of the patient, by way of a blood brain barrier (BBB) of the brain.

For some applications, the method includes sensing a parameter of the patient and presenting the odorant responsive thereto. The parameter may include an indication of a behavior of the patient, in which case sensing the parameter includes sensing the indication of the behavior of the patient. Alternatively, the parameter may be selected from the list consisting of: a biochemical value of the patient and a physiological value of the patient, in which case sensing the parameter includes sensing the parameter selected from the list. For some applications, sensing the parameter selected from the list includes sensing the parameter using a modality selected from the list consisting of: CT, MRI, PET, SPECT, angiography, ophthalmoscopy, fluoroscopy, light microscopy, and oximetry. Alternatively or additionally, sensing the parameter selected from the list includes measuring a level of the molecules in the patient. For some applications, measuring the level of the molecules includes sampling a body fluid of the patient selected from the list consisting of: blood, plasma, serum, ascites fluid, and urine.

In an embodiment of the present invention, presenting the odorant to the air passage of the patient includes presenting the odorant, the odorant having been selected for presentation to the air passage because it is such as to increase conductance of the molecules from the systemic blood circulation of the patient through the blood brain barrier (BBB) into brain tissue of the patient, the molecules being selected from the group consisting of: an endogenous agent, a pharmacological agent, a therapeutic agent, and, an agent for facilitating a diagnostic procedure.

In an embodiment, presenting the odorant includes presenting the odorant in a dosage determined to increase the conductance of the molecules. In an embodiment, the method includes administering the molecules for inhalation by the patient.

In an embodiment, the method includes administering the molecules to the patient in a bolus. In an embodiment, the method includes administering the molecules to the patient in a generally continuous manner.

In an embodiment, the method includes administering an agent capable of blocking a P-glycoprotein transporter from transporting the molecules from a target site in the brain tissue.

In an embodiment, the method includes administering the molecules to the systemic blood circulation. For some applications, administering the molecules includes administering the molecules mixed with the odorant. Alternatively or additionally, administering the molecules includes administering the molecules to the systemic blood circulation using a technique selected from the list consisting of: per-oral administration intravenous administration, intra-arterial administration, intraperitoneal administration, subcutaneous administration, and intramuscular administration.

In an embodiment, the molecules include the agent for facilitating a diagnostic procedure, and presenting the odorant includes presenting the odorant, the odorant being such as to increase the conductance of the agent for facilitating the diagnostic procedure. For some applications, the agent for facilitating a diagnostic procedure includes an imaging contrast agent, and presenting the odorant includes presenting the odorant, the odorant being such as to increase the conductance of the imaging contrast agent. Alternatively or additionally, the agent for facilitating a diagnostic procedure includes a radio-opaque material, and presenting the odorant includes presenting the odorant, the odorant being such as to increase the conductance of the radio-opaque material. Further alternatively or additionally, the agent for facilitating a diagnostic procedure includes an antibody, and presenting the odorant includes presenting the odorant, the odorant being such as to increase the conductance of the antibody.

In an embodiment, presenting the odorant includes selecting the molecules, the molecules being appropriate for treating a disorder of the central nervous system (CNS) of the patient. In an embodiment, the CNS disorder is selected from the list consisting of: a brain tumor, epilepsy, Parkinson's disease, Alzheimer's disease, multiple sclerosis, schizophrenia, depression, stress, obesity, pain, and anxiety, and selecting the molecules includes selecting the molecules, the molecules being appropriate for treating the selected CNS disorder.

In an embodiment, the method includes regulating a parameter of the odorant presentation. For some applications, regulating the parameter includes regulating a parameter selected from the list consisting of: relative concentrations of two or more ingredients of the odorant, a quantity of the odorant presented, a rate of presentation of the odorant, a pressure of the odorant at presentation, and a temperature of at least a portion of the odorant. In an embodiment, the method includes administering the molecules to the patient during a treatment session that is subsequent to regulating the parameter of the odorant presentation. In an embodiment, the method includes administering the molecules to the patient during a treatment session, and regulating the parameter of the odorant presentation during the same treatment session. For some applications, regulating the parameter of the odorant presentation includes selecting the parameter from a predefined set of parameters for the odorant presentation.

For some applications, the method includes sensing a parameter of the patient and regulating the parameter of the odorant presentation responsive thereto. The parameter of the patient may include an indication of a behavior of the patient, in which case sensing the parameter of the patient includes sensing the indication of the behavior of the patient Alternatively, the parameter of the patient may be selected from the list consisting of: a biochemical value of the patient and a physiological value of the patient, in which case sensing the parameter of the patient includes sensing the parameter of the patient selected from the list.

In an embodiment, the molecules include the therapeutic agent, and presenting the odorant includes presenting the odorant, the odorant being such as to increase the conductance of the therapeutic agent. For some applications, the therapeutic agent includes a neurological drug, and presenting the odorant includes presenting the odorant, the odorant being such as to increase the conductance of the neurological drug. For some applications, the therapeutic agent includes a protein, and presenting the odorant includes presenting the odorant, the odorant being such as to increase the conductance of the protein. For some applications, the therapeutic agent includes a polymer, and presenting the odorant includes presenting the odorant, the odorant being such as to increase the conductance of the polymer. For some applications, the therapeutic agent includes a viral vector, and presenting the odorant includes presenting the odorant, the odorant being such as to increase the conductance of the viral vector.

For some applications, the therapeutic agent includes an anti-cancer drug, and presenting the odorant includes presenting the odorant, the odorant being such as to increase the conductance of the anti-cancer drug. For some applications, the therapeutic agent includes an agent from the list consisting of: glatiramer acetate and interferon beta-1b, and presenting the odorant includes presenting the odorant, the odorant being such as to increase the conductance of the agent selected from the list. For some applications, the therapeutic agent includes an agent from the list consisting of: an agent for DNA therapy and an agent for RNA therapy, and presenting the odorant includes presenting the odorant, the odorant being such as to increase the conductance of the agent selected from the list. For some applications, the therapeutic agent includes an agent from the list consisting of: (a) an antisense molecule against type-1 insulin-like growth factor receptor, and (b) ADV-HSV-tk, and presenting the odorant includes presenting the odorant, the odorant being such as to increase the conductance of the agent selected from the list consisting of the antisense molecule and the ADV-HSV-tk.

In an embodiment, the method includes administering the molecules in conjunction with presenting the odorant. In an embodiment, administering the molecules in conjunction with presenting the odorant includes administering the molecules at a time determined with respect to a time of presenting the odorant. For some applications, administering the molecules includes administering the molecules at least a predetermined time prior to presenting the odorant. Alternatively, administering the molecules includes administering the molecules at generally the same time as presenting the odorant. Further alternatively, administering the molecules includes administering the molecules at least a predetermined time subsequent to presenting the odorant.

In an embodiment, the molecules include the pharmacological agent, and presenting the odorant includes presenting the odorant, the odorant being such as to increase the conductance of the pharmacological agent. For some applications, the pharmacological agent includes a viral vector, and presenting the odorant includes presenting the odorant, the odorant being such as to increase the conductance of the viral vector. For some applications, the pharmacological agent includes an antibody, and presenting the odorant includes presenting the odorant, the odorant being such as to increase the conductance of the antibody. For some applications, the antibody is selected from the list consisting of: a toxin-antibody complex, a radiolabeled antibody, and anti-HER2 mAb, and presenting the odorant includes presenting the odorant, the odorant being such as to increase the conductance of the selected antibody. Alternatively, the antibody is selected from the list consisting of: anti-b-amyloid antibody and anti-amyloid-precursor-protein antibody, and presenting the odorant includes presenting the odorant, the odorant being such as to increase the conductance of the selected antibody.

In an embodiment, the molecules include the endogenous agent, and presenting the odorant includes presenting the odorant, the odorant being such as to increase the conductance of the endogenous agent. For some applications, the endogenous agent includes an endogenous agent substantially unmodified by artificial means, and presenting the odorant includes presenting the odorant, the odorant being such as to increase the conductance of the endogenous agent that is substantially unmodified by artificial means. Alternatively, the endogenous agent includes an endogenous agent an aspect of which is modified by artificial means, and presenting the odorant includes presenting the odorant, the odorant being such as to increase the conductance of the endogenous agent the aspect of which is modified by artificial means. Further alternatively, the endogenous agent includes an enzyme, and presenting the odorant includes presenting the odorant, the odorant being such as to increase the conductance of the enzyme. For some applications, the enzyme includes hexosamimidase, and presenting the odorant includes presenting the odorant, the odorant being such as to increase the conductance of the hexosamimidase.

In an embodiment, the method includes administering the molecules to a mucous membrane of the patient. For some applications, administering the molecules includes administering the molecules to oral mucosa of the patient. Alternatively, administering the molecules includes administering the molecules to nasal mucosa of the patient.

For some applications, administering the molecules includes administering the molecules in combination with the odorant. Alternatively, administering the molecules includes administering the molecules separately from the odorant.

In an embodiment of the present invention, presenting the odorant to the air passage of the patient includes presenting the odorant, the odorant having been selected for presentation to the air passage because it is such as to increase conductance of molecules from the brain tissue of the patient through the blood brain barrier (BBB) into the systemic blood circulation.

In an embodiment, the method includes sensing a quantity of the molecules from a site outside of the brain of the patient, following initiation of presentation of the odorant. For some applications, sensing the quantity of the molecules includes sensing using a modality selected from the list consisting of: CT, MRI, PET, SPECT, angiography, ophthalmoscopy, fluoroscopy, light microscopy, and oximetry. For some applications, sensing the quantity of the molecules includes sampling a fluid of the patient selected from the list consisting of: blood, plasma, serum, ascites fluid, and urine.

In an embodiment, the method includes determining a diagnostically-relevant parameter responsive to sensing the quantity of the molecules.

In an embodiment, the method includes selecting a dosage of the odorant responsive to a disorder of the patient. For some applications, selecting the dosage of the odorant includes determining a dosage of the odorant that increases conductance of the molecules, responsive to presentation of the odorant, to an extent sufficient to treat the disorder at least in part. For some applications, selecting the dosage includes selecting the dosage responsive to the disorder of the patient, the disorder being selected from the list consisting of: a brain tumor, epilepsy, Parkinson's disease, Alzheimer's disease, multiple sclerosis, schizophrenia, depression, stress, obesity, pain, and anxiety.

In an embodiment, the method includes administering a hyperosmolarity-inducing agent to the patient at a dosage sufficient to augment an increase in conductance of the molecules caused by presentation of the odorant.

In an embodiment, the method includes inducing a state of dehydration of the patient, of an extent sufficient to augment an increase in conductance of the molecules caused by presentation of the odorant.

In an embodiment, the method includes administering an agent to the patient that modulates synthesis or metabolism of nitric-oxide (NO) in blood vessels of the brain, at a dosage sufficient to augment an increase in conductance of the molecules caused by presentation of the odorant.

There is additionally provided, in accordance with a preferred embodiment of the present invention, a method for modifying a property of a brain of a patient during or following a stroke event, including presenting an odorant to an air passage of the patient, the odorant having been selected for presentation to the air passage because it is capable of inducing an increase in cerebral blood flow of the patient, so as to reduce a pathology associated with the stroke event.

In an embodiment, presenting the odorant includes presenting the odorant in a dosage determined to increase the cerebral blood flow.

There is also provided, in accordance with a preferred embodiment of the present invention, a method for modifying a property of a brain of a patient who suffers from headache attacks, including presenting an odorant to an air passage of the patient, the odorant having been selected for presentation to the air passage because it is capable of modifying cerebral blood flow of the patient, so as to reduce a severity of a headache attack of the patient.

In an embodiment, presenting the odorant includes presenting the odorant in a dosage determined to modify the cerebral blood flow.

In an embodiment, presenting the odorant includes selecting the odorant, the odorant being capable of decreasing the cerebral blood flow, so as to reduce the severity of the headache attack.

In an embodiment, the headache attack includes a migraine headache attack of the patient, and presenting the odorant includes presenting to the air passage an odorant that is capable of reducing the cerebral blood flow, so as to reduce the severity of the migraine headache attack. In an embodiment, the headache attack includes a cluster headache attack of the patient, and presenting the odorant includes presenting to the air passage an odorant that is capable of reducing the cerebral blood flow, so as to reduce the severity of the cluster headache attack.

There is further provided, in accordance with a preferred embodiment of the present invention, a method for modifying a property of a brain of a patient who suffers from a disorder of the central nervous system (CNS), including presenting an odorant to an air passage of the patient, the odorant having been selected for presentation to the air passage because it is capable of modifying cerebral blood flow of the patient, so as to treat the CNS disorder.

In an embodiment, presenting the odorant includes presenting the odorant in a dosage determined to modify the cerebral blood flow.

In an embodiment, the CNS disorder is selected from the list consisting of: a brain tumor, epilepsy, Parkinson's disease, Alzheimer's disease, multiple sclerosis, schizophrenia, depression, stress, obesity, pain, and anxiety, and presenting the odorant includes presenting the odorant that is capable of modifying the cerebral blood flow, so as to treat the selected CNS disorder.

In an embodiment, presenting the odorant includes selecting the odorant, the odorant being capable of decreasing the cerebral blood flow. In an embodiment, presenting the odorant includes selecting the odorant, the odorant being capable of increasing cerebral blood flow of the patient. In an embodiment, presenting the odorant includes selecting the odorant, the odorant being capable of increasing cortical blood flow of the patient.

There is still further provided, in accordance with a preferred embodiment of the present invention, a method for modifying a property of a brain of a patient, including presenting an odorant to an air passage of the patient, the odorant having been selected for presentation to the air passage because it is such as to decrease conductance of molecules from a systemic blood circulation of the patient through a blood brain barrier (BBB) of the brain into brain tissue of the patient.

In an embodiment, presenting the odorant includes presenting the odorant in a dosage determined to decrease the conductance of the molecules.

In an embodiment, the method includes presenting in association with the odorant an analgesic in a dosage configured to reduce a sensation associated with the presenting of the odorant. In an embodiment, presenting the analgesic includes topically presenting the analgesic at a site selected from the list consisting of: a vicinity of one or more nerves in a nasal cavity of the patient, a vicinity of one or more nerves in an oral cavity of the patient, and a vicinity of one or more nerves innervating a face of the patient. In an embodiment, presenting the analgesic includes topically presenting the analgesic in a vicinity of a sphenopalatine ganglion (SPG) of the patient. In an embodiment, presenting the analgesic includes administering the analgesic for inhalation at generally the same time as the presenting of the odorant.

In an embodiment, the air passage includes a nasal cavity of the patient, and presenting the odorant includes presenting the odorant to the nasal cavity.

In an embodiment, the air passage includes a throat of the patient, and presenting the odorant includes presenting the odorant to the throat.

In an embodiment, the odorant is selected from the list consisting of: propionic acid, cyclohexanone, and amyl acetate, and presenting the odorant includes presenting the selected odorant. Alternatively, the odorant is selected from the list consisting of: acetic acid, citric acid, carbon dioxide, sodium chloride, and ammonia, and presenting the odorant includes presenting the selected odorant. Further alternatively, the odorant is selected from the list consisting of: menthol, alcohol, nicotine, piperine, gingerol, zingerone, allyl isothiocyanate, cinnamaldehyde, cuminaldehyde, 2-propenyl/2-phenylethyl isothiocyanate, thymol, and eucalyptol, and presenting the odorant includes presenting the selected odorant.

In an embodiment, presenting the odorant includes presenting a capsule for placement within a mouth of the patient, the capsule being configured to dissolve upon contact with salivary liquids of the patient, whereupon the odorant is presented to the air passage.

In an embodiment, the method includes regulating a parameter of the odorant presentation. For some applications, regulating the parameter includes regulating a parameter selected from the list consisting of: relative concentrations of two or more ingredients of the odorant, a quantity of the odorant presented, a rate of presentation of the odorant, a pressure of the odorant at presentation, and a temperature of at least a portion of the odorant. Alternatively or additionally, regulating the parameter of the odorant presentation includes selecting the parameter from a predefined set of parameters for the odorant presentation.

In an embodiment, the method includes sensing a parameter of the patient and regulating the parameter of the odorant presentation responsive thereto. For some applications, the parameter of the patient includes an indication of a behavior of the patient, and sensing the parameter of the patient includes sensing the indication of the behavior of the patient

In an embodiment, the parameter of the patient is selected from the list consisting of: a biochemical value of the patient and a physiological value of the patient, and sensing the parameter of the patient includes sensing the parameter of the patient selected from the list.

In an embodiment, the method includes sensing a parameter of the patient and presenting the odorant responsive thereto. For some applications, the parameter includes an indication of a behavior of the patient, and sensing the parameter includes sensing the indication of the behavior of the patient. Alternatively, the parameter is selected from the list consisting of: a biochemical value of the patient and a physiological value of the patient, and sensing the parameter includes sensing the parameter selected from the list. For some applications, sensing the parameter selected from the list includes sensing the parameter using a modality selected from the list consisting of: CT, MRI, PET, SPECT, angiography, ophthalmoscopy, fluoroscopy, light microscopy, and oximetry. Alternatively, sensing the parameter selected from the list includes sampling a body fluid of the patient selected from the list consisting of: blood, plasma, serum, ascites fluid, and urine.

There is additionally provided, in accordance with a preferred embodiment of the present invention, apparatus for modifying a property of a brain of a patient, including:
an odorant-storage vessel;
an odorant for storage within the odorant-storage vessel, the odorant being capable of increasing conductance of molecules from a systemic blood circulation of the patient through a blood brain barrier (BBB) of the brain into brain tissue of the patient, the molecules being selected from the group consisting of: a pharmacological agent, a therapeutic agent, and an agent for facilitating a diagnostic procedure; and
an odorant-delivery element, adapted to present the odorant to an air passage of the patient.

In an embodiment, the odorant-storage vessel is adapted to store the odorant mixed with the molecules.

In an embodiment, the molecules include the therapeutic agent, and the odorant is such as to increase the conductance of the therapeutic agent.

In an embodiment, the therapeutic agent includes a neurological drug, and the odorant is such as to increase the conductance of the neurological drug.

In an embodiment, the molecules include the agent for facilitating a diagnostic procedure, and the odorant is such as to increase the conductance of the agent for facilitating the diagnostic procedure. For some applications, the agent for facilitating a diagnostic procedure includes a radio-opaque material, and the odorant is such as to increase the conductance of the radio-opaque material.

In an embodiment, the odorant includes an agent for facilitating treatment of a disorder of the central nervous system (CNS) of the patient. For some applications, the CNS disorder is selected from the list consisting of: a brain tumor, epilepsy, Parkinson's disease, Alzheimer's disease, multiple sclerosis, schizophrenia, depression, stress, obesity, pain, and anxiety, and the odorant includes an agent for facilitating treatment of the selected CNS disorder.

There is yet additionally provided, in accordance with a preferred embodiment of the present invention, apparatus for modifying a property of a brain of a patient during or following a stroke event, including:
an odorant-storage vessel;
an odorant, for storage within the odorant-storage vessel, the odorant being capable of inducing an increase in cerebral blood flow of the patient; and
an odorant-delivery element, adapted to present the odorant to an air passage of the patient, so as to reduce a pathology associated with the stroke event.

There is further provided, in accordance with a preferred embodiment of the present invention, apparatus for modifying a property of a brain of a patient who suffers from headache attacks, including:
an odorant-storage vessel;
an odorant, for storage within the odorant-storage vessel, the odorant being capable of modifying cerebral blood flow of the patient; and
an odorant-delivery element, configured to present the odorant to an air passage of the patient, so as to reduce a severity of a headache attack of the patient.

In an embodiment, the odorant is capable of decreasing the cerebral blood flow.

In an embodiment, the headache attack includes a migraine headache attack of the patient, and the odorant is capable of reducing the severity of the migraine headache attack. In an embodiment, the headache attack includes a cluster headache attack of the patient, and the odorant is capable of reducing the severity of the cluster headache attack.

There is still additionally provided, in accordance with a preferred embodiment of the present invention, apparatus for modifying a property of a brain of a patient who suffers from a disorder of the central nervous system (CNS), including:
an odorant-storage vessel;
an odorant for storage within the odorant-storage vessel, the odorant being capable of modifying cerebral blood flow of the patient; and
an odorant-delivery element, configured to present the odorant to an air passage of the patient, so as to treat the CNS disorder.

In an embodiment, the CNS disorder is selected from the list consisting of: a brain tumor, epilepsy, Parkinson's disease, Alzheimer's disease, multiple sclerosis, schizophrenia, depression, stress, obesity, pain, and anxiety, and the odorant includes an agent for facilitating treatment of the selected CNS disorder.

In an embodiment, the odorant is capable of decreasing the cerebral blood flow. Alternatively, the odorant is capable of increasing the cerebral blood flow. For some applications, the odorant is capable of increasing cortical blood flow of the patient.

There is further provided, in accordance with a preferred embodiment of the present invention, apparatus for modifying a property of a brain of a patient, including:
an odorant-storage vessel;
an odorant, for storage within the odorant-storage vessel, the odorant being capable of decreasing conductance of molecules from a systemic blood circulation of the patient through a blood brain barrier (BBB) of the brain into brain tissue of the patient; and
an odorant-delivery element, adapted to present the odorant to an air passage of the patient.

In an embodiment, the apparatus includes an analgesic for storage within the odorant-storage vessel in a dosage configured to reduce a sensation associated with the presenting of the odorant, and the odorant-delivery element is adapted to present the analgesic to the air passage in association with the odorant.

In an embodiment, the odorant-storage vessel in combination with the odorant-delivery element includes an aqueous spray nasal inhaler. Alternatively, the odorant-storage vessel in combination with the odorant-delivery element includes a metered dose nasal inhaler. Further alternatively, the odorant-storage vessel in combination with the odorant-delivery element includes an air-dilution olfactometer.

In an embodiment, the air passage includes a nasal cavity of the patient, and the odorant-delivery element is adapted to present the odorant to the nasal cavity.

In an embodiment, the air passage includes a throat of the patient, and the odorant-delivery element is adapted to present the odorant to the throat.

In an embodiment, the odorant includes an agent selected from the list consisting of: propionic acid, cyclohexanone, and amyl acetate. Alternatively, the odorant includes an agent selected from the list consisting of: acetic acid, citric acid, carbon dioxide, sodium chloride, and anunonia. Further alternatively, the odorant includes an agent selected from the list consisting of: menthol, alcohol, nicotine, piperine, gingerol, zingerone, allyl isothiocyanate, cinnamaldehyde, cuminaldehyde, 2-propenyl/2-phenylethyl isothiocyanate, thymol, and eucalyptol.

In an embodiment, the odorant-storage vessel includes a capsule for placement in a mouth of the patient, and the odorant-delivery element includes a portion of the capsule adapted to dissolve upon contact with salivary liquids of the patient, whereupon the odorant is presented to the air passage of the patient.

The present invention will be more fully understood from the following detailed description of the preferred embodiments thereof, taken together with the drawings, in which:

### BRIEF DESCRIPTION OF THE DRAWINGS

FIG. 1 is a schematic pictorial view of a fully implantable stimulator for stimulation of the SPG, in accordance with a preferred embodiments of the present invention;

FIG. 2 is a schematic pictorial view of another stimulator for stimulation of the SPG, in accordance with a preferred embodiment of the present invention;

FIG. 3 is a schematic block diagram illustrating circuitry for use with the stimulator shown in FIG. 1, in accordance with a preferred embodiment of the present invention;

FIG. 4 is a schematic block diagram illustrating circuitry for use with the stimulator shown in FIG. 2, in accordance with a preferred embodiment of the present invention;

FIGS. 5A and 5B are schematic illustrations depicting different modes of operation of stimulators such as those shown in FIGS. 1 and 2, in accordance with preferred embodiments of the present invention;

FIG. 6 is a schematic illustration of a mode of operation of the stimulators shown in FIGS. 1 and 2, synchronized with a drug delivery system, in accordance with a preferred embodiment of the present invention;

FIG. 7 is a schematic black diagram illustrating circuitry for use with the stimulator shown in FIG. 1, where the stimulator is driven by an external controller and energy source using a modulator and a demodulator, in accordance with a preferred embodiment of the present invention;

FIG. 8 depicts sample modulator and demodulator functions for use with the circuitry of FIG. 7, in accordance with a preferred embodiment of the present invention;

FIGS. 9, 10A, and 10B are schematic diagrams illustrating further circuitry for use with implantable stimulators, in accordance with respective preferred embodiments of the present invention;

FIGS. 11 and 12 are bar graphs showing experimental data collected in accordance with a preferred embodiment of the present invention;

FIG. 13 is a schematic illustration of a sensor for application to a blood vessel, in accordance with a preferred embodiment of the present invention; and

FIG. 14 is a schematic sectional illustration of a nasal inhaler, for use in presenting an odorant to a subject, in accordance with a preferred embodiment of the present invention.

### DETAILED DESCRIPTION OF THE INVENTION

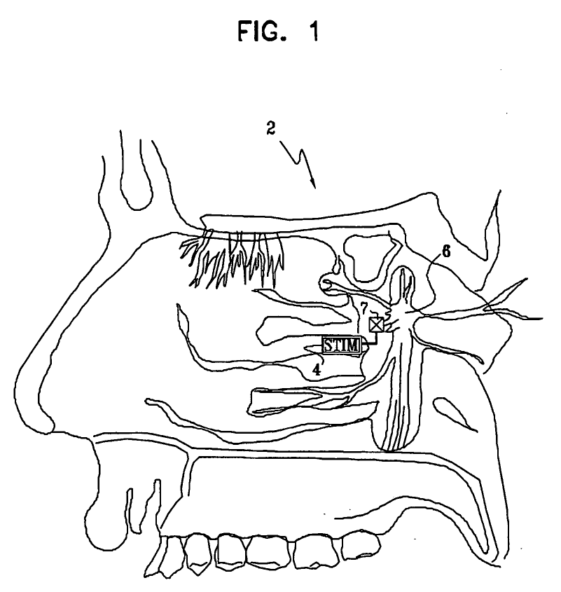

FIG. 1 is a schematic pictorial view of a fully-implantable stimulator 4, for stimulation of the sphenopalatine ganglion (SPG) 6 or other parasympathetic site of a patient, in accordance with a preferred embodiments of the present invention. In FIG. 1, a human nasal cavity 2 is shown, and stimulator 4 is implanted adjacent to SPG 6. Branches of parasympathetic neurons coming from SPG 6 extend to the middle cerebral and anterior cerebral arteries (not shown). Preferably, one or more relatively short electrodes 7 extend from stimulator 4 to contact or to be in a vicinity of SPG 6 or of nerves innervating SPG 6 (e.g., postganglionic parasympathetic trunks thereof).

For some applications, stimulator 4 is implanted on top of the bony palate, in the bottom of the nasal cavity. Alternatively or additionally, the stimulator is implanted at the lower side of the bony palate, at the top of the oral cavity. In this instance, one or more flexible electrodes 7 originating in the stimulator are passed through the palatine bone or posterior to the soft palate, so as to be in a position to stimulate the SPG or its parasympathetic tracts. Further alternatively or additionally, the stimulator may be directly attached to the SPG and/or to its postganglionic parasympathetic trunk(s).

For some applications, stimulator 4 is delivered to a desired point within nasal cavity 2 by removably attaching stimulator 4 to the distal end of a rigid or slightly flexible introducer rod (not shown) and inserting the rod into one of the patient's nasal passages until the stimulator is properly positioned. As appropriate, the placement process may be facilitated by fluoroscopy, x-ray guidance, fine endoscopic surgery (FES) techniques or by any other effective guidance method known in the art, or by combinations of the aforementioned. Preferably, the ambient temperature and/or cerebral blood flow is measured concurrently with insertion. The cerebral blood flow may be measured with, for example, a laser Doppler unit positioned at the patient's forehead or transcranial Doppler measurements. Verification of proper implantation of the electrodes onto the appropriate neural structure may be performed by activating the device, and generally simultaneously monitoring cerebral blood flow.

The passage of certain molecules from cerebral blood vessels into the brain is hindered by the BBB. The endothelium of the capillaries, the plasma membrane of the blood vessels, and the foot processes of the astrocytes all impede uptake by the brain of the molecules. The BBB generally allows only small molecules (e.g., hydrophilic molecules of molecular weight less than about 200 Da, and lipophilic molecules of less than about 500 Da) to pass from the circulation into the brain.

In accordance with a preferred embodiment of the present invention, parasympathetic activation induced by current from stimulator 4 overcomes the resistance to trans-BBB molecular movement generated by the endothelium of the cerebral capillaries and the plasma membrane. For some applications, therefore, stimulator 4 may be used to transiently remove a substantial obstacle to the passage of drugs from the blood to the brain. For example, the stimulator may cyclically apply current for about two minutes, and subsequently have a rest period of between about 1 and 20 minutes.

It is hypothesized that two neurotransmitters play an important role in this change in properties of the BBB—vasoactive intestinal polypeptide (VIP) and nitric oxide (NO). (Acetylcholine may also be involved.) VIP is a short peptide, and NO is a gaseous molecule. VIP is believed to be a major factor in facilitating plasma protein extravasation (PPE), while NO is responsible for vasodilation. For some applications, stimulator 4 is adapted to vary parameters of the current applied to the SPG, as appropriate, in order to selectively influence the activity of one or both of these neurotransmitters. For example, stimulation of the parasympathetic nerve at different frequencies can induce differential secretion—low frequencies cause secretion of NO, while high frequencies (e.g., above about 10 Hz) cause secretion of peptides (VIP).

For other applications, a constant level DC signal, or a slowly varying voltage ramp is applied, in order to block parasympathetic neural activity in affected tissue. Alternatively, similar results can be obtained by stimulating at a rate higher than about 10 Hz, because this tends to exhaust neurotransmitters. Thus, stimulator 4 may be configured to induce parasympathetic electrical block, in order to cause vasoconstriction by mimicking the overall effect of chemical block on the SPG. This vasoconstrictive effect may be used, for example, to controllably prevent or reverse the formation of migraine headaches. This technique of electrical treatment of migraines stands in contrast to methods of the prior art, in which pharmacological agents such as lidocaine are applied so as to induce SPG block.

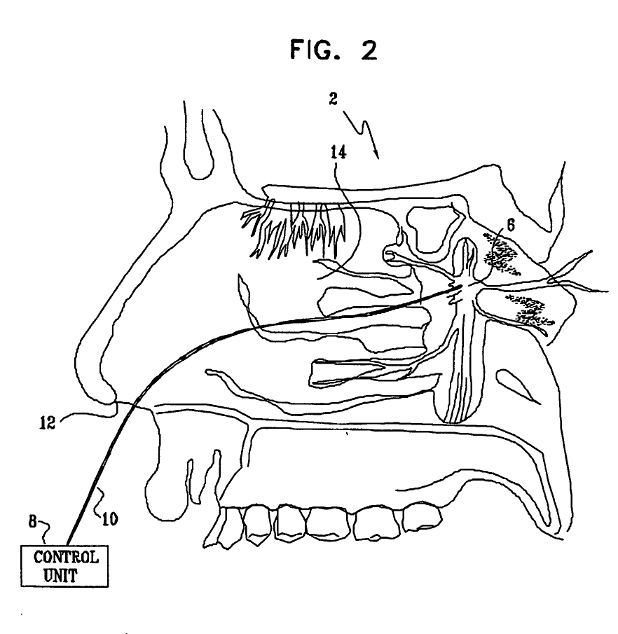

FIG. 2 is a schematic illustration of a stimulator control unit 8 positioned external to a patient's body, in accordance with a preferred embodiment of the present invention. At least one flexible electrode 10 preferably extends from control unit 8, through a nostril 12 of the patient, and to a position within the nasal cavity 14 that is adjacent to SPG 6.

It is to be understood that electrodes 7 (FIG. 1) and 10 may each comprise one or more electrodes, e.g., two electrodes, or an array of microelectrodes. For applications in which stimulator 4 comprises a metal housing that can function as an electrode, then typically one electrode 7 is used, operating in a monopolar mode. Regardless of the total number of electrodes in use, typically only a single or a double electrode extends to SPG 6. Other electrodes 7 or 10 or a metal housing of stimulator 4 are preferably temporarily or permanently implanted in contact with other parts of nasal cavity 2.

Each of electrodes 7 and/or 10 preferably comprises a suitable conductive material, for example, a physiologically-acceptable material such as silver, iridium, platinum, a platinum iridium alloy, titanium, nitinol, or a nickel-chrome alloy. For some applications, one or more of the electrodes have lengths ranging from about 1 to 5 mm, and diameters ranging from about 50 to 100 microns. Each electrode is preferably insulated with a physiologically-acceptable material such as polyethylene, polyurethane, or a co-polymer of either of these. The electrodes are preferably spiral in shape, for better contact, and may have a hook shaped distal end for hooking into or near the SPG. Alternatively or additionally, the electrodes may comprise simple wire electrodes, spring-loaded “crocodile” electrodes, or adhesive probes, as appropriate.

In a preferred embodiment of the invention, each one of electrodes 7 and/or 10 comprises a substantially smooth surface, except that the distal end of each such electrode is configured or treated to have a large surface area. For example, the distal tip may be porous platinized. Alternatively or additionally, at least the tip of electrode 7 or 10, and/or a metal housing of stimulator 4 includes a coating comprising an anti-inflammatory drug, such as beclomethasone sodium phosphate or beclomethasone phosphate. Alternatively, such an anti-inflammatory drug is injected or otherwise applied.

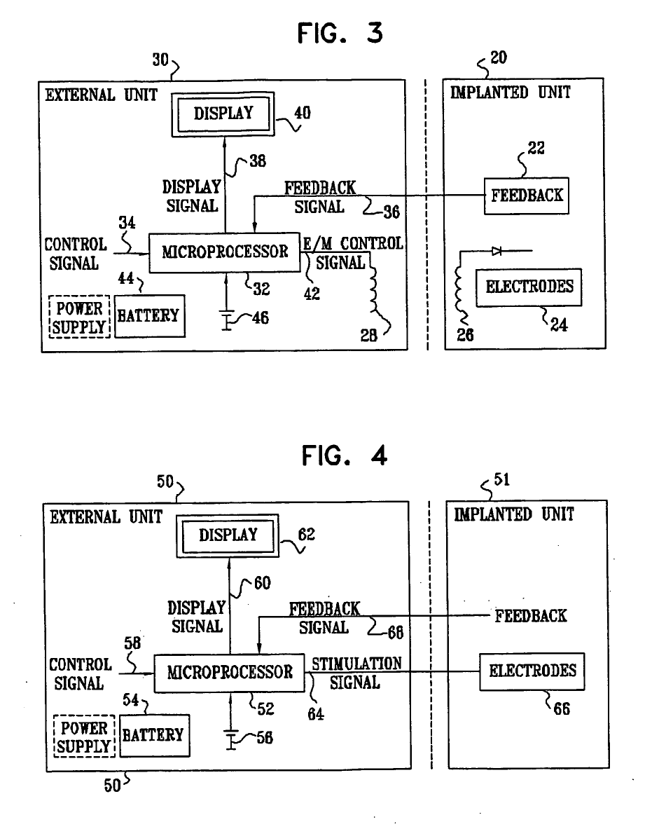

FIG. 3 is a schematic block diagram illustrating circuitry comprising an implanted unit 20 and an external unit 30, for use with stimulator 4 (FIG. 1), in accordance with a preferred embodiment of the present invention. Implanted unit 20 preferably comprises a feedback block 22 and one or more sensing or signal application electrodes 24. Implanted unit 20 typically also comprises an electromagnetic coupler 26, which receives power and/or sends or receives data signals to or from an electromagnetic coupler 28 in external unit 30.

External unit 30 preferably comprises a microprocessor 32 which receives an external control signal 34 (e.g., from a physician or from the patient), and a feedback signal 36 from feedback block 22. Control signal 34 may include, for example, operational parameters such as a schedule of operation, patient parameters such as the patient's weight, or signal parameters, such as desired frequencies or amplitudes of a signal to be applied to the SPG. If appropriate, control signal 34 can comprise an emergency override signal, entered by the patient or a healthcare provider to terminate stimulation or to modify it in accordance with a predetermined program. Microprocessor 32, in turn, preferably processes control signal 34 and feedback signal 36 so as to determine one or more parameters of the electric current to be applied through electrodes 24. Responsive to this determination, microprocessor 32 typically generates an electromagnetic control signal 42 that is conveyed by electromagnetic coupler 28 to electromagnetic coupler 26. Control signal 42 preferably corresponds to a desired current or voltage to be applied by electrodes 24 to SPG 6, and, in a preferred embodiment, inductively drives the electrodes. The configuration of couplers 26 and 28 and/or other circuitry in units 20 or 30 may determine the intensity, frequency, shape, monophasic or biphasic mode, or DC offset of the signal (e.g., a series of pulses) applied to designated tissue.

Power for microprocessor 32 is typically supplied by a battery 44 or, optionally, another DC power supply. Grounding is provided by battery 44 or a separate ground 46. If appropriate, microprocessor 32 generates a display signal 38 that drives a display block 40 of external unit 30. Typically, but not necessarily, the display is activated to show feedback data generated by feedback block 22, or to provide a user interface for the external unit.

Implanted unit 20 is preferably packaged in a case made of titanium, platinum or an epoxy or other suitable biocompatible material. Should the case be made of metal, then the case may serve as a ground electrode and, therefore, stimulation typically is performed in a monopolar mode. Alternatively, should the case be made of biocompatible plastic material, two electrodes 24 are typically driven to apply current to the SPG.

For some applications, the waveform applied by one or more of electrodes 24 to designated tissue (e.g., the SPG) comprises a waveform with an exponential decay, a ramp up or down, a square wave, a sinusoid, a saw tooth, a DC component, or any other shape known in the art to be suitable for application to tissue. Alternatively or additionally, the waveform comprises one or more bursts of short shaped or square pulses—each pulse preferably less than about 1 ms in duration. Generally, appropriate waveforms and parameters thereof are determined during an initial test period of external unit 30 and implanted unit 20. For some applications, the waveform is dynamically updated according to measured physiological parameters, measured during a period in which unit 20 is stimulating the SPG, and/or during a non-activation (i.e., standby) period.

In the case of migraine treatment, the waveform may take the form of a slowly varying shape, such as a slow saw tooth, or a constant DC level, intended to block outgoing parasympathetic messaging.

FIG. 4 is a schematic block diagram of circuitry for use, for example, in conjunction with control unit 8 (FIG. 2), in accordance with a preferred embodiment of the present invention. An external unit 50 comprises a microprocessor 52 supplied by a battery 54 or another DC power source. Grounding may be provided by battery 54 or by a separate ground 56. Microprocessor 52 preferably receives control and feedback signals 58 and 68 (analogous to signal 34 and 36 described hereinabove), and generates responsive thereto a stimulation signal 64 conveyed by one or more electrodes 66 to the SPG or other tissue. Typically, but not necessarily, feedback signal 68 comprises electrical feedback measured by one or more of electrodes 66 and/or feedback from other sensors on or in the patient's brain or elsewhere coupled to the patient's body. If appropriate, microprocessor 52 generates a display signal 60 which drives a display block 62 to output relevant data to the patient or the patient's physician. Typically, some or all of electrodes 66 are temporarily implanted in the patient (e.g., following a stroke), and are directly driven by wires connecting the external unit to the implanted unit.

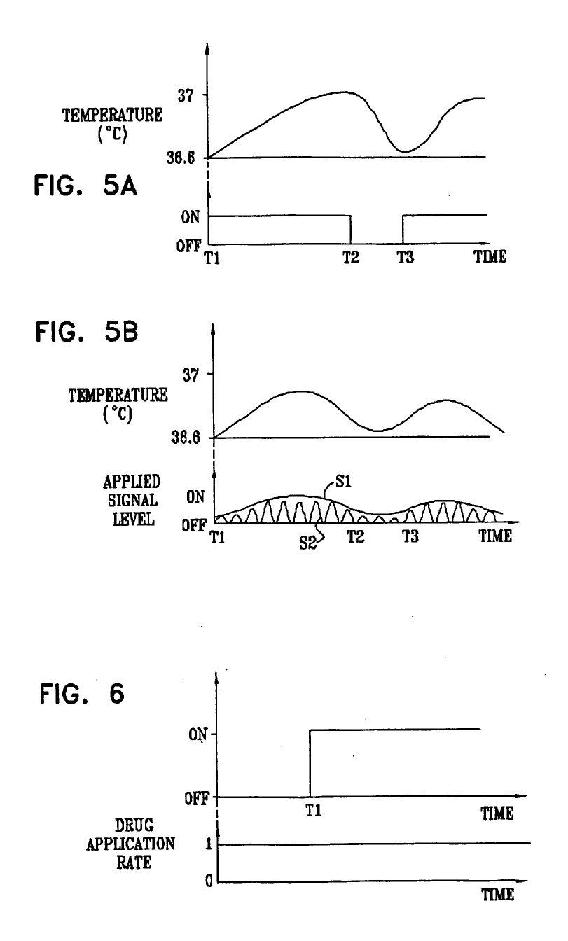

FIG. 5A is a graph schematically illustrating a mode of operation of one or more of the devices shown in FIGS. 1-4, in accordance with a preferred embodiment of the present invention. Preferably, the effect of the applied stimulation is monitored by means of a temperature transducer at the SPG or elsewhere in the head, e.g., in the nasal cavity. As shown in FIG. 5A for a step (ON/OFF) mode of stimulation, stimulation of the SPG or related tissue is initiated at a time T1, and this is reflected by a measurable rise in temperature (due to increased blood flow). Once the temperature rises to a predetermined or dynamically-varying threshold (e.g., 37° C.), stimulation is terminated (time T2), responsive to which the temperature falls. As appropriate, when the temperature drops to a designated or dynamically-determined point, the stimulation is reinitiated (time T3). Preferably, suitable temperatures or other physiological parameters are determined for each patient so as to provide the optimal treatment. If appropriate, control instructions may also be received from the patient, e.g., to initiate stimulation upon the onset of a migraine headache.

FIG. 5B is a graph schematically illustrating a mode of operation of one or more of the devices shown in FIGS. 1-4, in accordance with another preferred embodiment of the present invention. In this embodiment, the amplitude of the waveform applied to the SPG is varied among a continuous set of values (S1), or a discrete set of values (S2), responsive to the measured temperature, in order to achieve the desired performance. It will be appreciated that other feedback parameters measured in the head (e.g., intracranial pressure and/or cerebral blood flow), as well as measured systemic parameters (e.g., heart rate) and subjective patient inputs (e.g., migraine pain=3/5) may be used in conjunction with or separately from temperature measurements, in order to achieve generally optimal performance of the implanted apparatus.

FIG. 6 is a graph schematically illustrating a mode of operation of one or more of the devices shown in FIGS. 1-4, in accordance with a preferred embodiment of the present invention. In this embodiment, a drug is administered to the patient at a constant rate, e.g., intravenously, prior to the initiation of stimulation of the SPG at time T1. Advantageously, this prior generation of heightened concentrations of the drug in the blood tends to provide relatively rapid transfer of the drug across the BBB and into the brain, without unnecessarily prolonging the enhanced permeability of the BBB while waiting for the blood concentration of the drug to reach an appropriate level. Alternatively, for some applications it is desirable to give a single injection of a bolus of the drug shortly before or after initiation of stimulation of the SPG. Typically, combined administration and stimulation schedules are determined by the patient's physician based on the biochemical properties of each drug targeted at the brain.

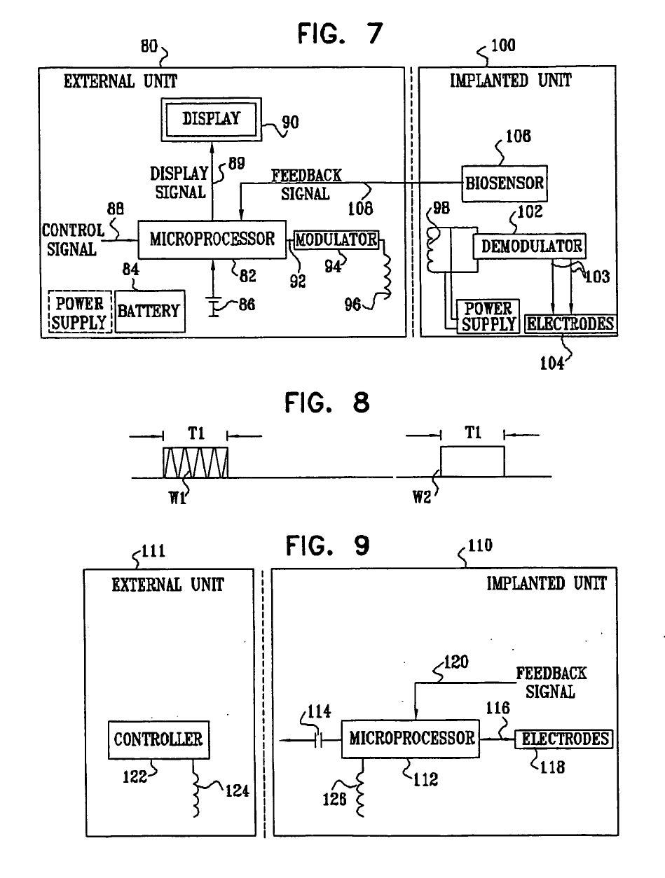

FIG. 7 is a schematic block diagram showing circuitry for parasympathetic stimulation, which is particularly useful in combination with the embodiment shown in FIG. 1, in accordance with a preferred embodiment of the present invention. An external unit 80 preferably comprises a microprocessor 82 that is powered by a battery 84 and/or an AC power source. Microprocessor 82 is grounded through battery 84 or through an optional ground 86.

In a typical mode of operation, an external control signal 88 is input to microprocessor 82, along with a feedback signal 108 from one or more biosensors 106, which are typically disposed in a vicinity of an implanted unit 100 or elsewhere on or in the patient's body. Responsive to signals 88 and 108, microprocessor 82 preferably generates a display signal 89 which drives a display 90, as described hereinabove. In addition, microprocessor 82 preferably processes external control signal 88 and feedback signal 108, to determine parameters of an output signal 92, which is modulated by a modulator 94. The output therefrom preferably drives a current through an electromagnetic coupler 96, which inductively drives an electromagnetic coupler 98 of implanted unit 100. A demodulator 102, coupled to electromagnetic coupler 98, in turn, generates a signal 103 which drives at least one electrode 104 to apply current to the SPG or to other tissue, as appropriate.

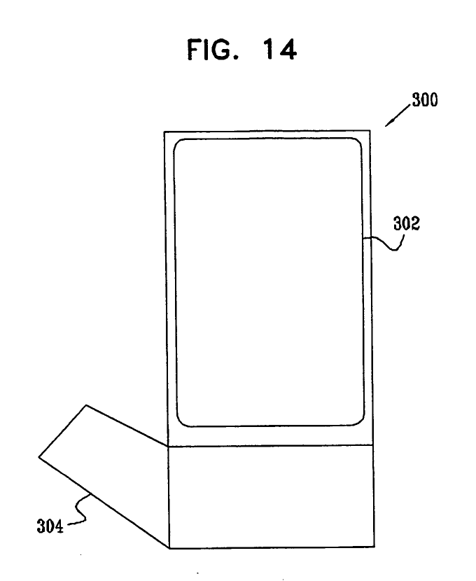

Preferably, biosensor 106 comprises implantable or external medical apparatus including, for example, one or more of the following:
a blood flow sensor,
a temperature sensor,
a chemical sensor,
an ultrasound sensor,
transcranial Doppler (TCD) apparatus,
laser-Doppler apparatus,
a systemic or intracranial blood pressure sensor (e.g., comprising a piezoelectric crystal fixed to a major cerebral blood vessel, capable of detecting a sudden blood pressure increase indicative of a clot),
a kinetics sensor, comprising, for example, an acceleration, velocity, or level sensor (e.g., a mercury switch), for indicating body dispositions such as a sudden change in body attitude (as in collapsing),
an electroencephalographic (EEG) sensor comprising EEG electrodes attached to, or implanted in, the patients head, for indicating changes in neurological patterns, such as symptoms of stroke or migraine,
a blood vessel clot detector (e.g., as described hereinbelow with reference to FIG. 13), or
other monitors of physiological quantities suitable for carrying out the objects of this or other embodiments of the present invention.

FIG. 8 is a schematic illustration showing operational modes of modulator 94 and/or demodulator 102, in accordance with a preferred embodiment of the present invention. The amplitude and frequency of signal 92 in FIG. 7 can have certain values, as represented in the left graph; however, the amplitude and frequency are modulated so that signal 103 has different characteristics.

FIG. 9 is a schematic illustration of further apparatus for stimulation of the SPG, in accordance with a preferred embodiment of the present invention. In this embodiment, substantially all of the processing and signal generation is performed by circuitry in an implanted unit 110 in the patient, and, preferably, communication with a controller 122 in an external unit 11 is performed only intermittently. The implanted unit 110 preferably comprises a microprocessor 112 coupled to a battery 114. Microprocessor 112 generates a signal 116 that travels along at least one electrode 118 to stimulate the SPG. A feedback signal 120 from a biosensor (not shown) and/or from electrode 118 is received by microprocessor 112, which is adapted to modify stimulation parameters responsive thereto. Preferably, microprocessor 112 and controller 122 are operative to communicate via electromagnetic couplers 126 and 124, in order to exchange data or to change parameters. Further preferably, battery 114 is inductively rechargeable by electromagnetic coupling.

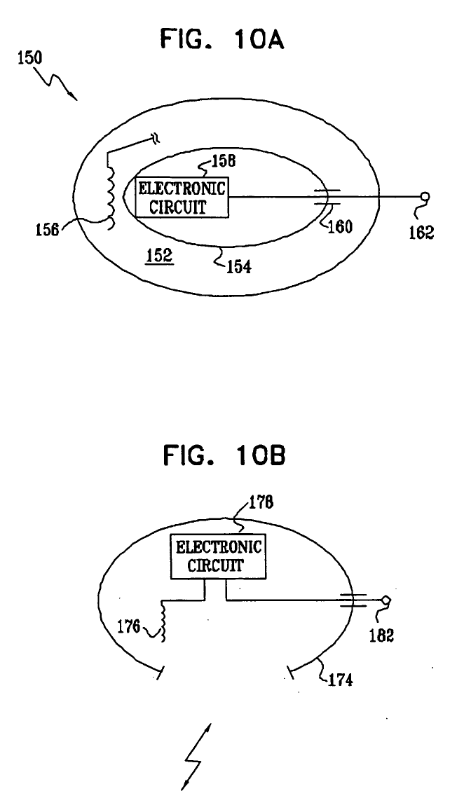

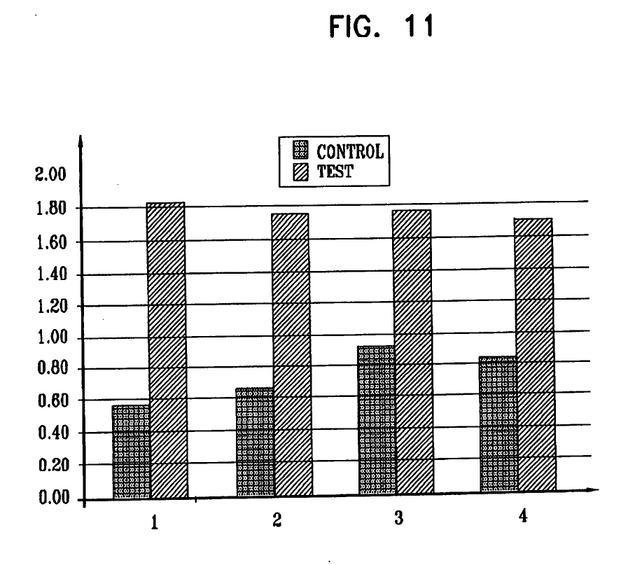

FIG. 10A is a schematic illustration of a stimulator 150, in accordance with a preferred embodiment of the present invention. Preferably, substantially all of the electronic components (including an electronic circuit 158 having a rechargeable energy source) are encapsulated in a biocompatible metal case 154. An inductive coil 156 and at least one electrode 162 are preferably coupled to circuit 158 by means of a feed-through coupling 160. The inductive coil is preferably isolated by an epoxy coating 152, which allows for higher efficiency of the electromagnetic coupling.

FIG. 10B is a schematic illustration of another configuration of an implantable stimulator, in accordance with a preferred embodiment of the present invention. Preferably, substantially all of the electronic components (including an inductive coil 176 and an electronic circuit 178 having a rechargeable energy source) are encapsulated in a biocompatible metal case 174. One or more feed-throughs are preferably provided to enable coupling between at least one electrode 182 and the electronic circuit, as well as between inductive coil 176 and another inductive coil (not shown) in communication therewith.

With reference to FIGS. 10A and 10B, the energy source for electronic circuits 158 and 178 may comprise, for example, a primary battery, a rechargeable battery, or a super capacitor. For applications in which a rechargeable battery or a super capacitor is used, any kind of energizing means may be used to charge the energy source, such as (but not limited to) standard means for inductive charging or a miniature electromechanical energy converter that converts the kinetics of the patient movement into electrical charge. Alternatively, an external light source (e.g., a simple LED, a laser diode, or any other light source) may be directed at a photovoltaic cell in the electronic circuit. Further alternatively, ultrasound energy is directed onto the implanted unit, and transduced to drive battery charging means.

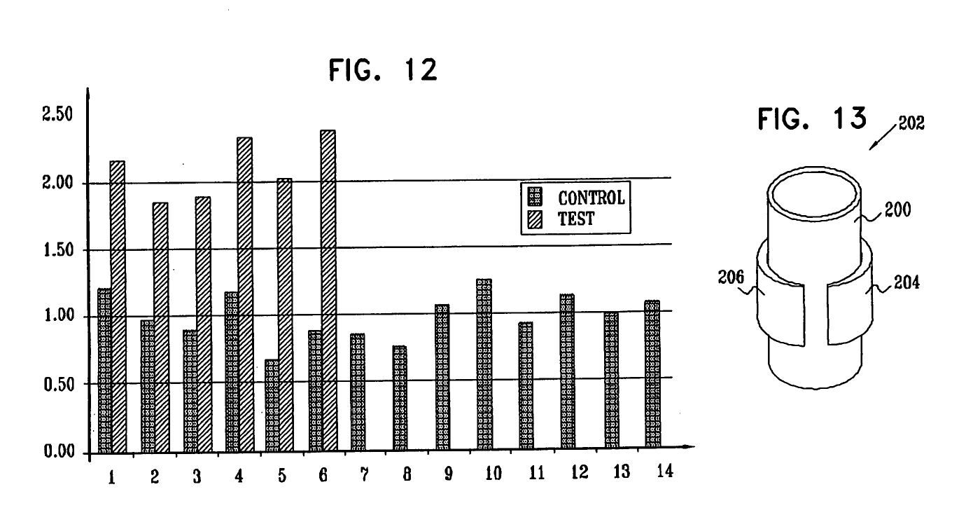

FIGS. 11 and 12 are bar graphs showing experimental results obtained during rat experiments performed in accordance with a preferred embodiment of the present invention. A common technique in monitoring bio-distribution of materials in a system includes monitoring the presence and level of radio-labeled tracers. These tracers are unstable isotopes of common elements (e.g., Tc, In, Cr, Ga, and Gd), conjugated to target materials. The chemical properties of the tracer are used as a predictor for the behavior of other materials with similar physiochemical properties, and are selected based on the particular biological mechanisms that are being evaluated. Typically, a patient or experimental animal is placed on a Gamma camera, or target tissue samples can be harvested and placed separately into a well counter. For the purpose of the present set of experiments which were performed, the well counter method was chosen due to its higher sensitivity and spatial resolution. A series of experiments using 99Tc-DTPA (DTPA molecule conjugated to a 99-Technetium isotope) were performed. The molecular weight of 99Tc-DTPA is 458 Da, its lipophilicity is negative, and its electric charge is +1. These parameters are quite similar with pharmacological agents used in standard chemotherapy, such as tamoxifen, etoposide and irinotecan.

FIGS. 11 and 12 show results obtained using 99Tc-DTPA penetration assays using ordinary brain sampling techniques (FIG. 11) and peeled brain techniques (FIG. 12). The x-axis of each graph represents different experimental runs, and the y-axis of each graph is defined as: [(hemisphere radioactivity)/(hemisphere weight)]/[(total injected radioactivity)/(total animal weight)]. The results obtained demonstrate an average 2.5-fold increase in the penetration of 99Tc-DTPA to the rat brain. It is noted that these results were obtained by unilateral stimulation of the SPG. The inventors believe that bilateral SPG stimulation will approximately double drug penetration, relative to unilateral SPG stimulation.

In both FIG. 11 and FIG. 12, some animals were designated as control animals, and other animals were designated as test animals. In each group, the left and right hemispheres were tested separately, and the height of each bar represents, for a given animal and a given hemisphere, the normalized level of radioactivity as defined above. Thus, FIG. 11 shows results from a total of four test hemispheres and four control hemispheres. FIG. 12 shows results from six test hemispheres and fourteen control hemispheres. The juxtaposition of control and test bars in the bar graphs is not meant to imply pairing of control and test hemispheres.

FIG. 13 is a schematic illustration of acoustic or optical clot detection apparatus 202, for use, for example, in providing feedback to any of the microprocessors or other circuitry described hereinabove, in accordance with a preferred embodiment of the present invention. The detection is preferably performed by coupling to a major blood vessel 200 (e.g., the internal carotid artery or aorta) a detecting element comprising an acoustic or optical transmitter/receiver 206, and an optional reflecting surface 204. Natural physiological liquids may serve as a mediating fluid between the device and the vessel. Preferably, the transmitter/receiver generates an ultrasound signal or electromagnetic signal which is reflected and returned, and a processor evaluates changes in the returned signal to detect indications of a newly-present clot. Alternatively, a transmitter is placed on side of the vessel and a receiver is placed on the other side of the vessel. In either case, for some applications, more than one such apparatus 202 are placed on the vessel, in order to improve the probability of successful clot detection for possible estimation of the clot's direction of motion within the vessel, and to lower the false alarm (i.e. false detection) rate.

FIG. 14 is a schematic sectional illustration of a nasal inhaler 300, for use in presenting an odorant to a subject, in accordance with a preferred embodiment of the present invention. Nasal inhaler 300 preferably comprises apparatus known in the art, such as an aqueous spray nasal inhaler, a metered dose nasal inhaler, or an air-dilution olfactometer. The odorant is stored in an odorant-storage vessel 302, and is delivered to a nasal passage using an odorant-delivery element 304, such as a nasal piece. Alternatively or additionally, the odorant is presented by means of an orally-dissolvable capsule that releases the active odorants upon contact with salivary liquids. The odorants reach the appropriate neural structures and induce vasodilatation, vasoconstriction and/or cerebrovascular permeability changes.

Embodiments of the present invention have many medical applications. For example, chemotherapeutic drugs need to pass into cerebral tissue in order to treat brain tumors. Most of the chemotherapeutic drugs have molecular weights of 200-1200 Da, and thus their transport through the blood-brain barrier (BBB) is highly restricted. To overcome the impedance of the BBB, an intracarotid infusion of high osmotic load has been used in the prior art in order to open the tight junctions of the BBB for a very short period (e.g., 25 minutes), during which the medications are given. This procedure is not simple—it is invasive, requires general anesthesia, requires subsequent intensive care, and is in any case relatively expensive. For these reasons, such intracarotid infusions are used only in very few healthcare facilities, even though some reports claim a substantial improvement in life expectancy in patients receiving chemotherapy in this manner.

Preferably, embodiments of the present invention which facilitate increased trans-BBB drug delivery, and therefore more efficient chemotherapy, also enable a reduction or elimination of the need for radiotherapy. It is noted that such irradiation of the brain is indicated in the literature to be a significant cause of long-term cognitive and other deficits.

The better delivery of drugs, as provided in accordance with a preferred embodiment of the present invention, is also a factor in the treatment of other disorders, such as Parkinson's disease, Alzheimer's disease, and other neurological diseases. For some applications, the trans-BBB delivery of various growth factors is facilitated using the techniques described herein. Growth factors are typically large molecules that stimulate the growth of neurons, and may be used to treat degenerative disorders, such as Parkinson's disease, Alzheimer's disease, and Motor Neuron Diseases (e.g., Lou Gehrig's disease).

Another preferred application of the present invention includes facilitating drug delivery across the BBB in order to treat inflammation in the brain, e.g., for cases of infectious diseases of the brain in immunocompromised patients. Similarly, medications to treat AIDS may be more effectively administered to sites in the brain through the BBB, when appropriate, through the use of methods and apparatus described herein. A further application of some embodiments of the present invention includes the delivery through the BBB of viruses that are agents of gene therapy (e.g., for treating Parkinson's disease). Similarly, methods and apparatus described herein may be used for metabolic disorders of the brain, such as GM2 gangliosidosis.

Another aspect of some preferred embodiments of the invention relates to the modulation of cerebral blood flow. Roughly 750,000 Americans suffer strokes each year. Stroke is the United States' third leading cause of death, killing about 160,000 Americans every year. More than 3 million people in the United States have survived strokes, of whom more than 2 million suffer crippling paralysis, speech loss and lapses of memory. About 85% of strokes are ischemic, i.e., a blood vessel is occluded and its territory is deprived of oxygen supply. A cerebral region that is totally deprived of blood supply is surrounded by a second region of partial lack of supply, whose vitality is at risk. This second region is one of the main targets of some embodiments of the invention—stimulation of the SPG will dilate its vessels and significantly improve that region's likelihood of survival. If the intervention is given early enough in the event (e.g., a few hours post-stroke), it might help also the core region of the stroke, as the thrombus is not yet organized, and dilation of the vessels may reintroduce blood supply to the tissue. Alternatively, SPG stimulation may allow the clot to move from a big vessel to a small vessel, and thus deprive blood supply only from a much smaller volume of the brain (which would, in any case, have probably been deprived of blood supply had the clot remained in place).

Population-based studies have shown that about 5% of men and 16% of women suffer migraine attacks. Over 80% of these people suffer some degree of headache-related disability. Parasympathetic block (in contrast to stimulation) is known to cause vasoconstriction. An embodiment of the present invention uses electrical means to induce the vasoconstrictive effect and treat migraine. For example, it may use techniques to block nerve messaging, such as applying a slowly-varying voltage, or in some cases, a constant level DC voltage.

Alzheimer's disease is becoming a major source of disability and financial load with the increase in life expectancy. In recent years, vascular factors have been considered prominent in the pathophysiology of the disease. Current therapy is generally concentrated along one line—cholinomimetic medications, which can, at most, slow down the deterioration of cognitive function in patients. SPG stimulation, as provided in accordance with a preferred embodiment of the present invention, is believed to increase blood flow and oxygen supply to the brain, and therefore help these patients. For this use, permanent stimulators may be implanted in the nasal cavity, for long-term intermittent stimulation.

Whereas some embodiments of the present invention are described herein with respect to enhancing permeability of the BBB so as to facilitate passage of molecules from the systemic circulation to brain tissue of a patient, this is by way of illustration and not limitation. In other embodiments, analogous techniques are utilized so as to facilitate enhanced clearance of molecules from brain tissue to the systemic circulation. For some applications, this enhanced clearance is utilized to facilitate a diagnostic procedure, for example by means of an imaging modality or a blood sample taken during or subsequent to increased BBB permeability. For other applications, the enhanced clearance of molecules is a goal in and of itself, for example in order to facilitate clearance of toxins from the brain.

Techniques described in this application may be practiced in combination with methods and apparatus described in one or more of the following patent applications, which are assigned to the assignee of the present patent application and are incorporated herein by reference:
PCT Publication WO 01/85094, filed May 7, 2001, entitled, “Method and apparatus for stimulating the sphenopalatine ganglion to modify properties of the BBB and cerebral blood flow”
US Provisional Patent Application 60/364,451, filed Mar. 15, 2002, entitled, “Applications of stimulating the sphenopalatine ganglion (SPG)”
U.S. Provisional Patent Application 60/368,657, filed Mar. 28, 2002, entitled, “SPG Stimulation”
U.S. Provisional Patent Application 60/376,048, filed Apr. 25, 2002, entitled, “Methods and apparatus for modifying properties of the BBB and cerebral circulation by using the neuroexcitatory and/or neuroinhibitory effects of odorants on nerves in the head”
U.S. Provisional Patent Application 60/388,931, filed Jun. 14, 2002, entitled “Methods and systems for management of Alzheimer's disease”
U.S. Provisional Patent Application 60/400,167, filed Jul. 31, 2002, entitled, “Delivering compounds to the brain by modifying properties of the BBB and cerebral circulation”
a US Provisional Patent Application, filed Nov. 14, 2002, entitled, “Surgical tools and techniques for sphenopalatine ganglion stimulation”
a US Provisional Patent Application, filed Nov. 14, 2002, entitled, “Stimulation circuitry and control of electronic medical device”
a US Patent Application, filed Nov. 14, 2002, entitled, “SPG stimulation for treating eye pathologies”
a US Patent Application, filed Nov. 14, 2002, entitled, “Administration of anti-inflammatory drugs into the CNS”
a US Provisional Patent Application, filed Nov. 14, 2002, entitled, “Stimulation for treating ear pathologies”
a US Provisional Patent Application, filed Feb. 20, 2003, entitled, “Stimulation for treating autoimmune-related disorders of the CNS”
a US Provisional Patent Application to Gross et al., filed Apr. 8, 2003, entitled, “Treating abnormal conditions of the mind and body by modifying properties of the blood-brain barrier and cephalic blood flow”

In particular, techniques of electrical signal application described in the above list of patent applications may be used together with or instead of odorant presentation. Thus, applications described herein which utilize odorant presentation may instead use electrical signal application to achieve generally similar results to those achieved through odorant presentation.

It is to be understood that the term “blood brain barrier (BBB),” as used in the context of the present patent application and in the claims, applies to the barrier between the systemic circulation and the brain as well as to the barrier between the systemic circulation and a tumor in the brain (sometimes referred to as the “blood tumor barrier”).

It will be appreciated by persons skilled in the art that the present invention is not limited to what has been particularly shown and described hereinabove. Rather, the scope of the present invention includes both combinations and subcombinations of the various features described hereinabove, as well as variations and modifications thereof that are not in the prior art, which would occur to persons skilled in the art upon reading the foregoing description. For example, elements which are shown in a figure to be housed within one integral unit may, for some applications, be disposed in a plurality of distinct units. Similarly, apparatus for communication and power transmission which are shown to be coupled in a wireless fashion may be, alternatively, coupled in a wired fashion, and apparatus for communication and power transmission which are shown to be coupled in a wired fashion may be, alternatively, coupled in a wireless fashion. In addition, it is to be understood that the scope of the present invention includes apparatus for carrying out methods described and/or claimed herein, and also includes methods corresponding to apparatus described and/or claimed herein.

1. A method for modifying a property of a brain of a patient, comprising presenting an odorant to an air passage of the patient, the odorant having been selected for presentation to the air passage because it is such as to increase conductance of molecules between a systemic blood circulation of the patient and brain tissue of the patient, by way of a blood brain barrier (BBB) of the brain.

2. A method according to claim 1, comprising sensing a parameter of the patient and presenting the odorant responsive thereto.

3. A method according to claim 2, wherein the parameter includes an indication of a behavior of the patient, and wherein sensing the parameter comprises sensing the indication of the behavior of the patient.

4. A method according to claim 2, wherein the parameter is selected from the list consisting of: a biochemical value of the patient and a physiological value of the patient, and wherein sensing the parameter comprises sensing the parameter selected from the list.

5. A method according to claim 4, wherein sensing the parameter selected from the list comprises sensing the parameter using a modality selected from the list consisting of: CT, MRI, PET, SPECT, angiography, ophthalmoscopy, fluoroscopy, light microscopy, and oximetry.

6. A method according to claim 4, wherein sensing the parameter selected from the list comprises measuring a level of the molecules in the patient.

7. A method according to claim 6, wherein measuring the level of the molecules comprises sampling a body fluid of the patient selected from the list consisting of: blood, plasma, serum, ascites fluid, and urine.

8. A method according to claim 1, wherein presenting the odorant to the air passage of the patient comprises presenting the odorant, the odorant having been selected for presentation to the air passage because it is such as to increase conductance of the molecules from the systemic blood circulation of the patient through the blood brain barrier (BBB) into brain tissue of the patient, the molecules being selected from the group consisting of: an endogenous agent, a pharmacological agent, a therapeutic agent, and an agent for facilitating a diagnostic procedure.

9. A method according to claim 8, wherein presenting the odorant comprises presenting the odorant in a dosage determined to increase the conductance of the molecules.

10. A method according to claim 8, comprising administering the molecules for inhalation by the patient.

11. A method according to claim 8, comprising administering the molecules to the patient in a bolus.

12. A method according to claim 8, comprising administering the molecules to the patient in a generally continuous manner.

13. A method according to claim 8, comprising administering an agent capable of blocking a P-glycoprotein transporter from transporting the molecules from a target site in the brain tissue.

14. A method according to claim 8, comprising administering the molecules to the systemic blood circulation.

15. A method according to claim 14, wherein administering the molecules comprises administering the molecules mixed with the odorant.

16. A method according to claim 14, wherein administering the molecules comprises administering the molecules to the systemic blood circulation using a technique selected from the list consisting of: per-oral administration intravenous administration, intra-arterial administration, intraperitoneal administration, subcutaneous administration, and intramuscular administration.

17. A method according to claim 8, wherein the molecules include the agent for facilitating a diagnostic procedure, and wherein presenting the odorant comprises presenting the odorant, the odorant being such as to increase the conductance of the agent for facilitating the diagnostic procedure.

18. A method according to claim 17, wherein the agent for facilitating a diagnostic procedure includes an imaging contrast agent, and wherein presenting the odorant comprises presenting the odorant, the odorant being such as to increase the conductance of the imaging contrast agent

19. A method according to claim 17, wherein the agent for facilitating a diagnostic procedure includes a radio-opaque material, and wherein presenting the odorant comprises presenting the odorant, the odorant being such as to increase the conductance of the radio-opaque material.

20. A method according to claim 17, wherein the agent for facilitating a diagnostic procedure includes an antibody, and wherein presenting the odorant comprises presenting the odorant, the odorant being such as to increase the conductance of the antibody.

21. A method according to claim 8, wherein presenting the odorant comprises selecting the molecules, the molecules being appropriate for treating a disorder of the central nervous system (CNS) of the patient.

22. A method according to claim 21, wherein the CNS disorder is selected from the list consisting of: a brain tumor, epilepsy, Parkinson's disease, Alzheimer's disease, multiple sclerosis, schizophrenia, depression, stress, obesity, pain, and anxiety, and wherein selecting the molecules comprises selecting the molecules, the molecules being appropriate for treating the selected CNS disorder.

23. A method according to claim 8, comprising regulating a parameter of the odorant presentation.

24. A method according to claim 23, wherein regulating the parameter comprises regulating a parameter selected from the list consisting of: relative concentrations of two or more ingredients of the odorant, a quantity of the odorant presented, a rate of presentation of the odorant, a pressure of the odorant at presentation, and a temperature of at least a portion of the odorant.

25. A method according to claim 23, comprising administering the molecules to the patient during a treatment session that is subsequent to regulating the parameter of the odorant presentation.

26. A method according to claim 23, comprising administering the molecules to the patient during a treatment session, and regulating the parameter of the odorant presentation during the same treatment session.

27. A method according to claim 23, wherein regulating the parameter of the odorant presentation comprises selecting the parameter from a predefined set of parameters for the odorant presentation.

28. A method according to claim 23, comprising sensing a parameter of the patient and regulating the parameter of the odorant presentation responsive thereto.

29. A method according to claim 28, wherein the parameter of the patient includes an indication of a behavior of the patient, and wherein sensing the parameter of the patient comprises sensing the indication of the behavior of the patient.

30. A method according to claim 28, wherein the parameter of the patient is selected from the list consisting of: a biochemical value of the patient and a physiological value of the patient, and wherein sensing the parameter of the patient comprises sensing the parameter of the patient selected from the list.

31. A method according to claim 8, wherein the molecules include the therapeutic agent, and wherein presenting the odorant comprises presenting the odorant, the odorant being such as to increase the conductance of the therapeutic agent.

32. A method according to claim 31, wherein the therapeutic agent includes a neurological drug, and wherein presenting the odorant comprises presenting the odorant, the odorant being such as to increase the conductance of the neurological drug.

33. A method according to claim 31, wherein the therapeutic agent includes a protein, and wherein presenting the odorant comprises presenting the odorant, the odorant being such as to increase the conductance of the protein.

34. A method according to claim 31, wherein the therapeutic agent includes a polymer, and wherein presenting the odorant comprises presenting the odorant, the odorant being such as to increase the conductance of the polymer.

35. A method according to claim 31, wherein the therapeutic agent includes a viral vector, and wherein presenting the odorant comprises presenting the odorant, the odorant being such as to increase the conductance of the viral vector.

36. A method according to claim 31, wherein the therapeutic agent includes an anti-cancer drug, and wherein presenting the odorant comprises presenting the odorant, the odorant being such as to increase the conductance of the anti-cancer drug.

37. A method according to claim 31, wherein the therapeutic agent includes an agent from the list consisting of: glatiramer acetate and interferon beta-1b, and wherein presenting the odorant comprises presenting the odorant, the odorant being such as to increase the conductance of the agent selected from the list.

38. A method according to claim 31, wherein the therapeutic agent includes an agent from the list consisting of: an agent for DNA therapy and an agent for RNA therapy, and wherein presenting the odorant comprises presenting the odorant, the odorant being such as to increase the conductance of the agent selected from the list.

39. A method according to claim 38, wherein the therapeutic agent includes an agent from the list consisting of: (a) an antisense molecule against type-1 insulin-like growth factor receptor, and (b) ADV-HSV-tk, and wherein presenting the odorant comprises presenting the odorant, the odorant being such as to increase the conductance of the agent selected from the list consisting of the antisense molecule and the ADV-HSV-tk.

40. A method according to claim 8, comprising administering the molecules in conjunction with presenting the odorant.

41. A method according to claim 40, wherein administering the molecules in conjunction with presenting the odorant comprises administering the molecules at a time determined with respect to a time of presenting the odorant.

42. A method according to claim 41, wherein administering the molecules comprises administering the molecules at least a predetermined time prior to presenting the odorant.

43. A method according to claim 41, wherein administering the molecules comprises administering the molecules at generally the same time as presenting the odorant.

44. A method according to claim 41, wherein administering the molecules comprises administering the molecules at least a predetermined time subsequent to presenting the odorant.

45. A method according to claim 8, wherein the molecules include the pharmacological agent, and wherein presenting the odorant comprises presenting the odorant, the odorant being such as to increase the conductance of the pharmacological agent.

46. A method according to claim 45, wherein the pharmacological agent includes a viral vector, and wherein presenting the odorant comprises presenting the odorant, the odorant being such as to increase the conductance of the viral vector.

47. A method according to claim 45, wherein the pharmacological agent includes an antibody, and wherein presenting the odorant comprises presenting the odorant, the odorant being such as to increase the conductance of the antibody.

48. A method according to claim 47, wherein the antibody is selected from the list consisting of: a toxin-antibody complex, a radiolabeled antibody, and anti-HER2 mAb, and wherein presenting the odorant comprises presenting the odorant, the odorant being such as to increase the conductance of the selected antibody.

49. A method according to claim 47, wherein the antibody is selected from the list consisting of: anti-b-amyloid antibody and anti-amyloid-precursor-protein antibody, and wherein presenting the odorant comprises presenting the odorant, the odorant being such as to increase the conductance of the selected antibody.

50. A method according to claim 8, wherein the molecules include the endogenous agent, and wherein presenting the odorant comprises presenting the odorant, the odorant being such as to increase the conductance of the endogenous agent.

51. A method according to claim 50, wherein the endogenous agent includes an endogenous agent substantially unmodified by artificial means, and wherein presenting the odorant comprises presenting the odorant, the odorant being such as to increase the conductance of the endogenous agent that is substantially unmodified by artificial means.

52. A method according to claim 50, wherein the endogenous agent includes an endogenous agent an aspect of which is modified by artificial means, and wherein presenting the odorant comprises presenting the odorant, the odorant being such as to increase the conductance of the endogenous agent the aspect of which is modified by artificial means.

53. A method according to claim 50, wherein the endogenous agent includes an enzyme, and wherein presenting the odorant comprises presenting the odorant, the odorant being such as to increase the conductance of the enzyme.

54. A method according to claim 53, wherein the enzyme includes hexosamimidase, and wherein presenting the odorant comprises presenting the odorant, the odorant being such as to increase the conductance of the hexosamimidase.

55. A method according to claim 8, comprising administering the molecules to a mucous membrane of the patient.

56. A method according to claim 55, wherein administering the molecules comprises administering the molecules to oral mucosa of the patient.

57. A method according to claim 55, wherein administering the molecules comprises administering the molecules to nasal mucosa of the patient.

58. A method according to claim 55, wherein administering the molecules comprises administering the molecules in combination with the odorant.

59. A method according to claim 55, wherein administering the molecules comprises administering the molecules separately from the odorant.

60. A method according to claim 1, wherein presenting the odorant to the air passage of the patient comprises presenting the odorant, the odorant having been selected for presentation to the air passage because it is such as to increase conductance of molecules from the brain tissue of the patient through the blood brain barrier (BBB) into the systemic blood circulation.

61. A method according to claim 60, comprising sensing a quantity of the molecules from a site outside of the brain of the patient, following initiation of presentation of the odorant.

62. A method according to claim 61, wherein sensing the quantity of the molecules comprises sensing using a modality selected from the list consisting of: CT, MRI, PET, SPECT, angiography, ophthalmoscopy, fluoroscopy, light microscopy, and oximetry.

63. A method according to claim 61, wherein sensing the quantity of the molecules comprises sampling a fluid of the patient selected from the list consisting of: blood, plasma, serum, ascites fluid, and urine.

64. A method according to claim 61, comprising determining a diagnostically-relevant parameter responsive to sensing the quantity of the molecules.

65. A method according to claim 60, comprising selecting a dosage of the odorant responsive to a disorder of the patient.

66. A method according to claim 65, wherein selecting the dosage of the odorant comprises determining a dosage of the odorant that increases conductance of the molecules, responsive to presentation of the odorant, to an extent sufficient to treat the disorder at least in part.

67. A method according to claim 65, wherein selecting the dosage comprises selecting the dosage responsive to the disorder of the patient, the disorder being selected from the list consisting of: a brain tumor, epilepsy, Parkinson's disease, Alzheimer's disease, multiple sclerosis, schizophrenia, depression, stress, obesity, pain, and anxiety.

68. A method according to any one of claims 1, 8, or 60, comprising administering a hyperosmolarity-inducing agent to the patient at a dosage sufficient to augment an increase in conductance of the molecules caused by presentation of the odorant.

69. A method according to any one of claims 1, 8, or 60, comprising inducing a state of dehydration of the patient, of an extent sufficient to augment an increase in conductance of the molecules caused by presentation of the odorant.

70. A method according to any one of claims 1, 8, or 60, comprising administering an agent to the patient that modulates synthesis or metabolism of nitric-oxide (NO) in blood vessels of the brain, at a dosage sufficient to augment an increase in conductance of the molecules caused by presentation of the odorant.

71. A method for modifying a property of a brain of a patient during or following a stroke event, comprising presenting an odorant to an air passage of the patient, the odorant having been selected for presentation to the air passage because it is capable of inducing an increase in cerebral blood flow of the patient, so as to reduce a pathology associated with the stroke event.

72. A method according to claim 71, wherein presenting the odorant comprises presenting the odorant in a dosage determined to increase the cerebral blood flow.

73. A method for modifying a property of a brain of a patient who suffers from headache attacks, comprising presenting an odorant to an air passage of the patient, the odorant having been selected for presentation to the air passage because it is capable of modifying cerebral blood flow of the patient, so as to reduce a severity of a headache attack of the patient.

74. A method according to claim 73, wherein presenting the odorant comprises presenting the odorant in a dosage determined to modify the cerebral blood flow.

75. A method according to claim 73, wherein presenting the odorant comprises selecting the odorant, the odorant being capable of decreasing the cerebral blood flow, so as to reduce the severity of the headache attack.

76. A method according to claim 73, wherein the headache attack includes a migraine headache attack of the patient, and wherein presenting the odorant comprises presenting to the air passage an odorant that is capable of reducing the cerebral blood flow, so as to reduce the severity of the migraine headache attack.

77. A method according to claim 73, wherein the headache attack includes a cluster headache attack of the patient, and wherein presenting the odorant comprises presenting to the air passage an odorant that is capable of reducing the cerebral blood flow, so as to reduce the severity of the cluster headache attack.

78. A method for modifying a property of a brain of a patient who suffers from a disorder of the central nervous system (CNS), comprising presenting an odorant to an air passage of the patient, the odorant having been selected for presentation to the air passage because it is capable of modifying cerebral blood flow of the patient, so as to treat the CNS disorder.

79. A method according to claim 78, wherein presenting the odorant comprises presenting the odorant in a dosage determined to modify the cerebral blood flow.

80. A method according to claim 78, wherein the CNS disorder is selected from the list consisting of: a brain tumor, epilepsy, Parkinson's disease, Alzheimer's disease, multiple sclerosis, schizophrenia, depression, stress, obesity, pain, and anxiety, and wherein presenting the odorant comprises presenting the odorant that is capable of modifying the cerebral blood flow, so as to treat the selected CNS disorder.

81. A method according to claim 78, wherein presenting the odorant comprises selecting the odorant, the odorant being capable of decreasing the cerebral blood flow.

82. A method according to claim 78, wherein presenting the odorant comprises selecting the odorant, the odorant being capable of increasing cerebral blood flow of the patient.

83. A method according to claim 82, wherein presenting the odorant comprises selecting the odorant, the odorant being capable of increasing cortical blood flow of the patient.

84. A method for modifying a property of a brain of a patient, comprising presenting an odorant to an air passage of the patient, the odorant having been selected for presentation to the air passage because it is such as to decrease conductance of molecules from a systemic blood circulation of the patient through a blood brain barrier (BBB) of the brain into brain tissue of the patient.

85. A method according to claim 84, wherein presenting the odorant comprises presenting the odorant in a dosage determined to decrease the conductance of the molecules.

86. A method according to any one of claims 1, 8, 60, 71, 73, 78, or 84, and comprising presenting in association with the odorant an analgesic in a dosage configured to reduce a sensation associated with the presenting of the odorant.

87. A method according to claim 86, wherein presenting the analgesic comprises topically presenting the analgesic at a site selected from the list consisting of: a vicinity of one or more nerves in a nasal cavity of the patient, a vicinity of one or more nerves in an oral cavity of the patient, and a vicinity of one or more nerves innervating a face of the patient.

88. A method according to claim 86, wherein presenting the analgesic comprises topically presenting the analgesic in a vicinity of a sphenopalatine ganglion (SPG) of the patient.

89. A method according to claim 86, wherein presenting the analgesic comprises administering the analgesic for inhalation at generally the same time as the presenting of the odorant.

90. A method according to any one of claims 1, 8, 60, 71, 73, 78, or 84, wherein the air passage includes a nasal cavity of the patient, and wherein presenting the odorant comprises presenting the odorant to the nasal cavity.

91. A method according to any one of claims 1, 8, 60, 71, 73, 78, or 84, wherein the air passage includes a throat of the patient, and wherein presenting the odorant comprises presenting the odorant to the throat.

92. A method according to any one of claims 1, 8, 60, 71, 73, 78, or 84, wherein the odorant is selected from the list consisting of: propionic acid, cyclohexanone, and amyl acetate, and wherein presenting the odorant comprises presenting the selected odorant.

93. A method according to any one of claims 1, 8, 60, 71, 73, 78, or 84, wherein the odorant is selected from the list consisting of: acetic acid, citric acid, carbon dioxide, sodium chloride, and ammonia, and wherein presenting the odorant comprises presenting the selected odorant.

94. A method according to any one of claims 1, 8, 60, 71, 73, 78, or 84, wherein the odorant is selected from the list consisting of: menthol, alcohol, nicotine, piperine, gingerol, zingerone, allyl isothiocyanate, cinnamaldehyde, cuminaldehyde, 2-propenyl/2-phenylethyl isothiocyanate, thymol, and eucalyptol, and wherein presenting the odorant comprises presenting the selected odorant.

95. A method according to any one of claims 1, 8, 60, 71, 73, 78, or 84, wherein presenting the odorant comprises presenting a capsule for placement within a mouth of the patient, the capsule being configured to dissolve upon contact with salivary liquids of the patient, whereupon the odorant is presented to the air passage.

96. A method according to any one of claims 1, 60, 71, 73, 78, or 84, comprising regulating a parameter of the odorant presentation.

97. A method according to claim 96, wherein regulating the parameter comprises regulating a parameter selected from the list consisting of: relative concentrations of two or more ingredients of the odorant, a quantity of the odorant presented, a rate of presentation of the odorant, a pressure of the odorant at presentation, and a temperature of at least a portion of the odorant.

98. A method according to claim 96, wherein regulating the parameter of the odorant presentation comprises selecting the parameter from a predefined set of parameters for the odorant presentation.

99. A method according to claim 96, comprising sensing a parameter of the patient and regulating the parameter of the odorant presentation responsive thereto.

100. A method according to claim 99, wherein the parameter of the patient includes an indication of a behavior of the patient, and wherein sensing the parameter of the patient comprises sensing the indication of the behavior of the patient.

101. A method according to claim 99, wherein the parameter of the patient is selected from the list consisting of: a biochemical value of the patient and a physiological value of the patient, and wherein sensing the parameter of the patient comprises sensing the parameter of the patient selected from the list.

102. A method according to any one of claims 71, 73, 78, or 84, comprising sensing a parameter of the patient and presenting the odorant responsive thereto.

103. A method according to claim 102, wherein the parameter includes an indication of a behavior of the patient, and wherein sensing the parameter comprises sensing the indication of the behavior of the patient.

104. A method according to claim 102, wherein the parameter is selected from the list consisting of: a biochemical value of the patient and a physiological value of the patient, and wherein sensing the parameter comprises sensing the parameter selected from the list.

105. A method according to claim 104, wherein sensing the parameter selected from the list comprises sensing the parameter using a modality selected from the list consisting of: CT, MRI, PET, SPECT, angiography, ophthalmoscopy, fluoroscopy, light microscopy, and oximetry.

106. A method according to claim 104, wherein sensing the parameter selected from the list comprises sampling a body fluid of the patient selected from the list consisting of: blood, plasma, serum, ascites fluid, and urine.

107. Apparatus for modifying a property of a brain of a patient, comprising:
an odorant-storage vessel;
an odorant for storage within the odorant-storage vessel, the odorant being capable of increasing conductance of molecules from a systemic blood circulation of the patient through a blood brain barrier (BBB) of the brain into brain tissue of the patient, the molecules being selected from the group consisting of: a pharmacological agent, a therapeutic agent, and an agent for facilitating a diagnostic procedure; and
an odorant-delivery element, adapted to present the odorant to an air passage of the patient.

108. Apparatus according to claim 107, wherein the odorant-storage vessel is adapted to store the odorant mixed with the molecules.

109. Apparatus according to claim 107, wherein the molecules include the therapeutic agent, and wherein the odorant is such as to increase the conductance of the therapeutic agent.

110. Apparatus according to claim 109, wherein the therapeutic agent includes a neurological drug, and wherein the odorant is such as to increase the conductance of the neurological drug.

111. Apparatus according to claim 107, wherein the molecules include the agent for facilitating a diagnostic procedure, and wherein the odorant is such as to increase the conductance of the agent for facilitating the diagnostic procedure.

112. Apparatus according to claim 111, wherein the agent for facilitating a diagnostic procedure includes a radio-opaque material, and wherein the odorant is such as to increase the conductance of the radio-opaque material.

113. Apparatus according to claim 107, wherein the odorant comprises an agent for facilitating treatment of a disorder of the central nervous system (CNS) of the patient.

114. Apparatus according to claim 113, wherein the CNS disorder is selected from the list consisting of: a brain tumor, epilepsy, Parkinson's disease, Alzheimer's disease, multiple sclerosis, schizophrenia, depression, stress, obesity, pain, and anxiety, and wherein the odorant comprises an agent for facilitating treatment of the selected CNS disorder.

115. Apparatus for modifying a property of a brain of a patient during or following a stroke event, comprising:
an odorant-storage vessel;
an odorant, for storage within the odorant-storage vessel, the odorant being capable of inducing an increase in cerebral blood flow of the patient; and
an odorant-delivery element, adapted to present the odorant to an air passage of the patient, so as to reduce a pathology associated with the stroke event.

116. Apparatus for modifying a property of a brain of a patient who suffers from headache attacks, comprising:
an odorant-storage vessel;
an odorant, for storage within the odorant-storage vessel, the odorant being capable of modifying cerebral blood flow of the patient; and
an odorant-delivery element, configured to present the odorant to an air passage of the patient, so as to reduce a severity of a headache attack of the patient.

117. Apparatus according to claim 116, wherein the odorant is capable of decreasing the cerebral blood flow.

118. Apparatus according to claim 116, wherein the headache attack includes a migraine headache attack of the patient, and wherein the odorant is capable of reducing the severity of the migraine headache attack.

119. Apparatus according to claim 116, wherein the headache attack includes a cluster headache attack of the patient, and wherein the odorant is capable of reducing the severity of the cluster headache attack.

120. Apparatus for modifying a property of a brain of a patient who suffers from a disorder of the central nervous system (CNS), comprising:
an odorant-storage vessel;
an odorant for storage within the odorant-storage vessel, the odorant being capable of modifying cerebral blood flow of the patient; and
an odorant-delivery element, configured to present the odorant to an air passage of the patient, so as to treat the CNS disorder.

121. Apparatus according to claim 120, wherein the CNS disorder is selected from the list consisting of: a brain tumor, epilepsy, Parkinson's disease, Alzheimer's disease, multiple sclerosis, schizophrenia, depression, stress, obesity, pain, and anxiety, and wherein the odorant comprises an agent for facilitating treatment of the selected CNS disorder.

122. Apparatus according to claim 120, wherein the odorant is capable of decreasing the cerebral blood flow.

123. Apparatus according to claim 120, wherein the odorant is capable of increasing the cerebral blood flow.

124. Apparatus according to claim 123, wherein the odorant is capable of increasing cortical blood flow of the patient.

125. Apparatus for modifying a property of a brain of a patient, comprising:
an odorant-storage vessel;
an odorant, for storage within the odorant-storage vessel, the odorant being capable of decreasing conductance of molecules from a systemic blood circulation of the patient through a blood brain barrier (BBB) of the brain into brain tissue of the patient; and
an odorant-delivery element, adapted to present the odorant to an air passage of the patient.

126. Apparatus according to any one of claims 107, 115, 116, 120, or 125, comprising an analgesic for storage within the odorant-storage vessel in a dosage configured to reduce a sensation associated with the presenting of the odorant, and wherein the odorant-delivery element is adapted to present the analgesic to the air passage in association with the odorant.

127. Apparatus according to any one of claims 107, 115, 116, 120, or 125, wherein the odorant-storage vessel in combination with the odorant-delivery element comprises an aqueous spray nasal inhaler.

128. Apparatus according to any one of claims 107, 115, 116, 120, or 125, wherein the odorant-storage vessel in combination with the odorant-delivery element comprises a metered dose nasal inhaler.

129. Apparatus according to any one of claims 107, 115, 116, 120, or 125, wherein the odorant-storage vessel in combination with the odorant-delivery element comprises an air-dilution olfactometer.

130. Apparatus according to any one of claims 107, 115, 116, 120, or 125, wherein the air passage includes a nasal cavity of the patient, and wherein the odorant-delivery element is adapted to present the odorant to the nasal cavity.

131. Apparatus according to any one of claims 107, 115, 116, 120, or 125, wherein the air passage includes a throat of the patient, and wherein the odorant-delivery element is adapted to present the odorant to the throat.

132. Apparatus according to any one of claims 107, 115, 116, 120, or 125, wherein the odorant comprises an agent selected from the list consisting of propionic acid, cyclohexanone, and amyl acetate.

133. Apparatus according to any one of claims 107, 115, 116, 120, or 125, wherein the odorant comprises an agent selected from the list consisting of: acetic acid, citric acid, carbon dioxide, sodium chloride, and ammonia.

134. Apparatus according to any one of claims 107, 115, 116, 120, or 125, wherein the odorant comprises an agent selected from the list consisting of: menthol, alcohol, nicotine, piperine, gingerol, zingerone, allyl isothiocyanate, cinnamaldehyde, cuminaldehyde, 2-propenyl/2-phenylethyl isothiocyanate, thymol, and eucalyptol.

135. Apparatus according to any one of claims 107, 115, 116, 120, or 125, wherein the odorant-storage vessel comprises a capsule for placement in a mouth of the patient, and wherein the odorant-delivery element comprises a portion of the capsule adapted to dissolve upon contact with salivary liquids of the patient, whereupon the odorant is presented to the air passage of the patient.
